SEQUENCE LISTING (21 sequences)

SEQ ID NO: 1 - DNA, 2915 bp, Homo sapiens
agccccgccgcgatgcccgcgcgcccaggacgcctcctcccgctgctggcccggccggcggccctgactgcgctgctgctgctgctgctgggccatggcggcggcgggcgctggggcgcccgggcccaggaggcggcggcggcggcggcggacgggccccccgcggcagacggcgaggacggacaggacccgcacagcaagcacctgtacacggccgacatgttcacgcacgggatccagagcgccgcgcacttcgtcatgttcttcgcgccctggtgtggacactgccagcggctgcagccgacttggaatgacctgggagacaaatacaacagcatggaagatgccaaagtctatgtggctaaagtggactgcacggcccactccgacgtgtgctccgcccagggggtgcgaggataccccaccttaaagcttttcaagccaggccaagaagctgtgaagtaccagggtcctcgggacttccagacactggaaaactggatgctgcagacactgaacgaggagccagtgacaccagagccggaagtggaaccgcccagtgcccccgagctcaagcaagggctgtatgagctctcagcaagcaactttgagctgcacgttgcacaaggcgaccactttatcaagttcttcgctccgtggtgtggtcactgcaaagccctggctccaacctgggagcagctggctctgggccttgaacattccgaaactgtcaagattggcaaggttgattgtacacagcactatgaactctgctccggaaaccaggttcgtggctatcccactcttctctggttccgagatgggaaaaaggtggatcagtacaagggaaagcgggatttggagtcactgagggagtacgtggagtcgcagctgcagcgcacagagactggagcgacggagaccgtcacgccctcagaggccccggtgctggcagctgagcccgaggctgacaagggcactgtgttggcactcactgaaaataacttcgatgacaccattgcagaaggaataaccttcatcaagttttatgctccatggtgtggtcattgtaggactctggctcctacttgggaggaactctctaaaaaggaattccctggtctggcgggggtcaagatcgccgaagtagactgcactgctgaacggaatatctgcagcaagtattcggtacgaggctaccccacgttattgcttttccgaggagggaagaaagtcagtgagcacagtggaggcagagaccttgactcgttacaccgctttgtcctgagccaagcgaaagacgaactttaggaacacagttggaggtcacctctcctgcccagctcccgcaccctgcgtttaggagttcagtcccacagaggccactgggttcccagtggtggctgttcagaaagcagaacatactaagcgtgaggtatcttctttgtgtgtgtgttttccaagccaacacactctacagattctttattaaatgtgtaactcatggtcactgtgtaaacattttcagtggcgatatatcccctttgaccttctcttgatgaaatttacatggtttcctttgagactaaaatagcgttgagggaaatgaaattgctggactatttgtggctcctgagttgagtgattttggtgaaagaaagcacatccaaagcatagtttacctgcccacgagttctggaaaggttgccttgtggcagtattgacgttcctctgatcttaaggtcacagttgactcaatactgtgttggtccgtagcatggagcagattgaaatgcaaaaacccacacctctggaggataccttcacggccgctgctggagcttctgttgctgtgaatacttctctcagtgtgagaggttagccgtgatgaaagcagcgttacttctgaccgtgcctgagtaagagaatgctgatgccataactttatgtgtcgatacttgtcaaatcagttactgttcaggggatccttctgtttctcacggggtgaaacatgtctttagttcctcatgttaacacgaagccagagcccacatgaactgttggatgtcttccttagaaagggtaggcatggaaaattccacgaggctcattctcagtatctcattaactcattgaaagattccagttgtatttgtcacctggggtgacaagaccagacaggctttcccaggcctgggtatccagggaggctctgcagccctgctgaagggccctaactagagttctagagtttctgattctgtttctcagtagtccttttagaggcttgctatacttggtctgcttcaaggaggtcgaccttctaatgtatgaagaatgggatgcatttgatctcaagaccaaagacagatgtcagtgggctgctctggccctggtgtgcacggctgtggcagctgttgatgccagtgtcctctaactcatgctgtccttgtgattaaacacctctatctcccttgggaataagcacatacaggcttaagctctaagatagataggtgtttgtccttttaccatcgagctacttcccataataaccactttgcatccaacactcttcacccacctcccatacgcaaggggatgtggatacttggcccaaagtaactggtggtaggaatcttagaaacaagaccacttatactgtctgtctgaggcagaagataacagcagcatctcgaccagcctctgccttaaaggaaatctttattaatcacgtatggttcacagataattctttttttaaaaaaacccaacctcctagagaagcacaactgtcaagagtcttgtacacacaacttcagctttgcatcacgagtcttgtattccaagaaaatcaaagtggtacaatttgtttgtttacactatgatactttctaaataaactcctttttttt

SEQ ID NO: 2 - protein, 432 aa, Homo sapiens [sequence omitted]

SEQ ID NO: 3 - protein, 432 aa, Homo sapiens
MPARPGRLLPLLARPAALTALLLLLLGHGGGGRWGARAQEAAAAAADGPPAADGEDGQDPHSKHLYTADMFTHGIQSAAHFVMFFAPWCGHCQRLQPTWNDLGDKYNSMEDAKVYVAKVDCTAHSDVCSAQGVRGYPTLKLFKPGQEAVKYQGPRDFQTLENWMLQTLNEEPVTPEPEVEPPSAPELKQGLYELSASNFELHVAQGDHFIKFFAPWCGHCKALAPTWEQLALGLEHSETVKIGKVDCTQHYELCSGNQVRGYPTLLWFRDGKKVDQYKGKRDLESLREYVESQLQRTETGATETVTPSEAPVLAAEPEADKGTVLALTENNFDDTIAEGITFIKFYAPWCGHCKTLAPTWEELSKKEFPGLAGVKIAEVDCTAERNICSKYSVRGYPTLLLFRGGKKVSEHSGGRDLDSLHRFVLSQAKDEL

SEQ ID NO: 4 - protein, 417 aa, Rattus norvegicus
MPPRPGRLFQPLAGLPALATLLLLLGARKGARAQEVEADSGVEQDPHAKHLYTADMFTHGIQSAAHFVMFFAPWCGHCQRLQPTWNDLGDKYNSMEDAKVYVAKVDCTANSDVCSAQGVRGYPTLKFFKPGQEAVKYQGPRDFETLENWMLQTLNEEPATPEPEAEPPRAPELKQGLYELSANNFELHVSQGNHFIKFFAPWCGHCKALAPTWEQLALGLEHSETVKIGKVDCTQHYAVCSEHQVRGYPTLLWFRDGKKVDQYKGKRDLESLRDYVQSQLQGSEAAPETVEPSEAPVLAAEPPGDKGTVLALTEKSFEDTIAQGITFVKFYAPWCGHCKNLAPTWEELSKKEFPGLADVTIAEVDCTAERGVCSKYSVRGYPTLLLFRGGEKVGEHNGGRDLDSLHSFVLRQAKDEL

SEQ ID NO: 5 - protein, 417 aa, Mus musculus
MPPRPGRLLQPLAGLPALATLLLLLGARKGARAQEVEADSGVEQDPHAKHLYTADMFTHGIQSAAHFVMFFAPWCGHCQRLQPTWNDLGDKYNSMEDAKVYVAKVDCTADSDVCSAQGVRGYPTLKFFKPGQEAVKYQGPRDFETLENWMLQTLNEEPATPEPEAEPPRAPELKQGLYELSANNFELHVSQGNHFIKFFAPWCGHCKALAPTWEQLALGLEHSETVKIGKVDCTQHYAVCSEHQVRGYPTLLWFRDGKKVDQYKGKRDLESLRDYVQSQLQGSEAAPETVEPSEAPVMAAEPTGDKGTVLALTEKSFEDTIAQGITFVKFYAPWCGHCKNLAPTWEELSKKEFPGLSDVTIAEVDCTAERNVCSKYSVRGYPTLLLFRGGEKVGEHNGGRDLDSLHSFVLRQAKDEL

SEQ ID NO: 6 - protein, 403 aa, Xenopus laevis
MLRCPSAVLLCALLLRVSGEPWEEGADDDPHKKNLYSADMFDHAVKQEPHFIMFFAPWCGHCQRLQSTWNELGDKYNTMPNTPAYIAKVDCTTDMPTCTNHGVRGYPTLKLFKPGQEAVKYQGPRDLQSLENWMLQTLNAEAEKPKVEEKAEDPAKVPELKQGLYELTGANFKEHVAEGYHFIKFFAPWCGHCKSLAPAWEQLAASFQDSKSVKIAKVDCTQHNELCSEYQVRGYPTLLWFRNGEKVDQYKGKRDLDTMKEYAESQLKPAEEKKEEEQKKEATPPQVQEPVESKVLSLSESNFDQTVATGVSFIKFYAPWCGHCKNLAPIWEDLAKKEFSGMSDVKIAKVDCTAERSVCSRFSVRGYPSLLLFRAGEKIGEHEGARDLETLQNYVLRHSRDEL

SEQ ID NO: 7 - protein, 416 aa, Drosophila melanogaster
MLTRSILSVAVCGLLLSPLLPITRASQEEDTGKQDKQFTVELDPETFDTAIAGGNVFVKFFAPWCGHCKRIQPLWEQLAEIMNVDNPKVIIAKVDCTKHQGLCATHQVTGYPTLRLFKLGEEESVKFKGTRDLPAITDFINKELSAPAEADLGEVKREQVENLNIGKVVDLTEDTFAKHVSTGNHFVKFFAPWCSHCQRLAPTWEDLAKELIKEPTVTISKIDCTQFRSICQDFEVKGYPTLLWIEDGKKIEKYSGARDLSTLKTYVEKMVGVPLEKTAGEAGDEKVVIEEVAGEEDAAKKLTPQQLTGEDEFDQAIAEGVAFIKFYAPWCGHCQKLQPTWEQLATETHQAQSSVKIAKVDCTAPENKQVCIDQQVEGYPTLFLYKNGQRQNEYEGSRSLPELQAYLKKFLGHDEL

SEQ ID NO: 8 - protein, 424 aa, Anopheles gambiae
MAGMQRTIVGLMVAAALLATLASGHADTASVHLTKDNFQSELEGSSYFVMFYAPWCDYCKKLAPTWATLAKARNGDPDGVVKIGRVDCTTDGDLCTQHDVTGYPMLKLFRKDGGADGATKYRGARDLAQFNAFLDEQLAPAAGDGPAKGADRDGEDGAQEDGGQADAPPAPVSPLTELTEDTFAKHVSSGKHFVKFYAPWCGHCTKLAPTWEELARSLEHERDIRVSKIDCTQYRPICTDFEVKGYPTLLWIEDGKKIEKYTGPRTHADLKQYVARMAGGLKEDGAQGAEPKGEGTLEGGAERDDNRSVVVQLSEGDFAHAIAKGVTVVKFYAPWCGHCMRLAPTWEQLAEKLTARDGVTIAKVDCTVDANKELCGEQEVNGYPTVFLYRDGEKVTEYFGHRSLDDLHEFVMQHLQDNGPHDEL

SEQ ID NO: 9 - protein, 6 aa, Artificial sequence
  Motif
WCGHCK

SEQ ID NO: 10 - protein, 4 aa, Artificial sequence
  Motif
KDEL

SEQ ID NO: 11 - protein, 4 aa, Artificial sequence
  Motif
CGHC

SEQ ID NO: 12 - DNA, 33 bp, Artificial sequence
  Upstream primer
ccggtacccccgcgcgcccaggacgcctcctcc

SEQ ID NO: 13 - DNA, 20 bp, Artificial sequence
  Downstream primer
gcagcatccagttttccagt

SEQ ID NO: 14 - protein, 13 aa, Artificial sequence
  Synthetic sequence which corresponds to amino acids 52 to 63 of human EndoPI protein sequence with an additional Cys added to the C-terminus
ADGEDGQDPHSKC

SEQ ID NO: 15 - DNA, 20 bp, Artificial sequence
  Upstream forward primer
tgtggctcctgagttgagtg

SEQ ID NO: 16 - DNA, 20 bp, Artificial sequence
  Reverse downstream primer
actcaggcacggtcagaagt

SEQ ID NO: 17 - DNA, 21 bp, Artificial sequence
  EndoPDI specific siRNA oligo
gaagctgtgaagtaccaggtt

SEQ ID NO: 18 - DNA, 21 bp, Artificial sequence
  PDI specific siRNA oligo
gacctccccttcaaagttgtt

SEQ ID NO: 19 - protein, 7 aa, Artificial sequence
  Thioredoxin motif
APWCGHC

SEQ ID NO: 20 - protein, 34 aa, Homo sapiens
MPARPGRLLPLLARPAALTALLLLLLGHGGGGRW

SEQ ID NO: 21 - protein, 5 aa, Homo sapiens
GGGRW

(19) United States
(12) Patent Application Publication
(10) Pub. No.: US 2005/0153361 A1
(43) Pub. Date: Jul. 14, 2005

(54) Therapeutic and diagnostic applications of protein disulphide isomerases

(76) Inventors: Dianne C. Sullivan, Oxford (GB); Roy Bicknell, Oxford (GB)

Correspondence Address: NIXON & VANDERHYE, PC, 1100 N GLEBE ROAD, 8TH FLOOR, ARLINGTON, VA, 22201-4714, US

(21) Appl. No.: 10/935,752

(22) Filed: Sep. 8, 2004

Related U.S. Application Data
(60) Provisional application No. 60/501,032, filed on Sep. 9, 2003.

(51) Int. Cl. G01N 33/53

(52) U.S. Cl. 435/7.1; 436/518; 435/25

(57) ABSTRACT

An endothelial form of protein disulphide isomerase (endoPDI) is specifically upregulated in endothelial cells in response to hypoxia. Inhibition of endoPDI expression in hypoxic cells induces apoptosis. Thus the invention provides the use of endoPDI as a marker for angiogenesis and as a therapeutic target for the inhibition of angiogenesis. Agents capable of binding to or inhibiting endoPDI may be used for the detection and treatment of solid tumours.

The present application claims benefit of U.S. Provisional application Ser. No. 60/501,032, filed 9 Sep. 2003, the entire contents of which is incorporated herein by reference.

### FIELD OF THE INVENTION

The present invention relates to protein disulphide isomerases, and in particular to an endothelial form of protein disulphide isomerase and its use as a marker of angiogenesis.

### BACKGROUND TO THE INVENTION

Protein disulphide isomerase (PDI) is a ubiquitously expressed multifunctional protein found in the endoplasmic reticulum (ER). It constitutes around 0.8% of total cellular protein and can reach near millimolar concentrations in the ER lumen of some tissues. PDI plays a role in protein folding due to its ability to catalyse the formation of native,disulphide bonds and disulphide bond rearrangement (1). Proteins targetted for secretion by the cell are inserted into and translocated across the ER membrane and enter the ER lumen in an unfolded state. PDI, together with a variety of other folding factors and molecular chaperones resident in the ER correctly fold the proteins ready for secretion (2). The accumulation of misfolded proteins in the ER, known as the Unfolded Protein Response, results in increased transcription of chaperones and folding catalysts. Proteins that fail to fold correctly are relocated to the cytosol for proteosomal degradation.

PDI is a modular protein consisting of a, b, b′, a′ and c domains (3). The a and a′ domains show sequence and structural homology to thioredoxin (Trx) and both contain the active site WCGHCK motif, constituting two independent catalytic sites for thiol-disulphide bond exchange reactions (4-7). A rate-limiting step in the folding of many newly synthesized proteins is the formation of disulphide bridges (1) and the presence of WCGHCK in PDI is essential for this process, as confirmed by the loss of PDI activity following mutation of the cysteine residues within these motifs (5,8). The b and b′ domains also have the thioredoxin structural fold but lack the active site motif. Thus, PDI contains both redox active and inactive thioredoxin modules. The C-terminal c domain, a putative Ca2+ binding region, is rich in acidic amino acids and contains the —KDEL motif which is necessary and sufficient for the retention of a polypeptide within the lumen of the ER. The c-terminal domain is, however, not necessary for the enzymatic, chaperone (see below) or disulphide isomerase activities of PDI (9). In fact, the smallest PDI fragment showing an efficient catalysis of disulphide bond rearrangement has been shown to be a construct containing the b′-a′-c modules (7).

In addition to its disulphide isomerase activity, PDI also shows chaperone activity, for example it can function as the β-subunit of prolyl-4 hydroxylase, preventing the misfolding and aggregation of the α-subunit (10). This function is similar to that of some molecular chaperones such as Hsp90 in other proteins (1). Furthermore, PDI is able to interact with and correctly fold type X collagen polypeptides that contain no cysteine residues (11).

There is now an increasing family of protein disulphide isomerases, each having two or more thioredoxin or catalytically inactive ‘b’ domains (1,12). Sequence homology between members is poor, their relatedness lying in the structural similarity of the thioredoxin-like fold (12). It has been proposed that different PDIs may show different substrate specificities (1) and support for this has been provided in studies showing that ERp57 is specific for the folding of N-glycosylated proteins (13,14).

Recently a detailed microarray analysis by Claudio et al. (31) has identified a putative disulphide isomerase mRNA sequence (designated MGC3178 in that study) as being strongly upregulated in multiple myeloma. However these authors did not investigate whether the reported high level of expression is a cause or a consequence of the pathogenic state, and did not attempt to confirm the putative function of the protein or otherwise characterise it.

### SUMMARY OF THE INVENTION

Broadly, the present invention is based on the finding that endoPDI is specifically upregulated in endothelial cells in response to hypoxia, and that inhibition of endoPDI expression in such cells sensitises the cells to hypoxia, leading them to undergo apoptosis.

Growth of solid tumours is frequently characterised by angiogenesis, in which blood vessels are formed which supply nutrients and oxygen to the hypoxic centre of the cell mass. Inhibition of angiogenesis has been proposed in the art as a means of treating such tumours, by cutting off this supply of nutrients and oxygen. Other conditions are also characterised by angiogenesis, and may be treatable by inhibition of angiogenesis. The present invention proposes that the inhibition of activity or expression of endoPDI, for example in vascular endothelium, is useful for the selective inhibition of hypoxia-induced angiogenesis without damage to the existing vasculature. It also proposes the use of endoPDI as a marker for angiogenesis which allows the specific targeting of diagnostic and therapeutic agents to sites of angiogenesis. It further proposes the use of the transcriptional regulatory sequences of endoPDI for the targeted expression of nucleic acid sequences.

In a first aspect the present invention provides methods of screening for substances capable of modulating endoPDI activity or expression. Broadly, substances capable of stimulating endoPDI activity or expression will be referred to as endoPDI activators, while substances capable of inhibiting endoPDI activity or expression will be referred to as endoPDI antagonists.

Thus the present invention provides a method for testing the ability of a candidate substance to modulate endoPDI activity, comprising contacting endoPDI with the candidate substance and determining the effect of the candidate substance on endoPDI activity. The effect of the candidate substance on endoPDI activity may be determined quantitatively or qualitatively.

The method preferably comprises assessing formation or cleavage of a disulphide bond in or between one or more reporter molecules. The disulphide bond may be intramolecular (i.e. between two cysteine residues in the same molecule) or intermolecular (i.e. between two cysteine residues in different molecules).

The method may comprise assessing rearrangement of disulphide bonds, which involves both cleavage and formation of disulphide bonds.

In preferred embodiments, the specificity of the candidate substance towards endoPDI is tested by determining the effect of the candidate substance on one or more control proteins, typically also having a thioredoxin domain. Thus the method may comprise contacting a control protein comprising a thioredoxin domain with the candidate substance and determining the effect of the candidate substance on the activity of the control protein.

Preferably, the thioredoxin domain is catalytically active. Typically such thioredoxin domains comprise the motif CXXC, typically CGHC. Suitable control proteins possess protein disulphide isomerase activity, i.e. they are protein disulphide isomerases other than endoPDI, such as the archetypal human PDI (see below). Human PDIs are reviewed in detail by Clissold and Bicknell (12) and by Freedman et al. (39).

The method may comprise contacting endoPDI with a library comprising at least one candidate substance and selecting a substance capable of modulating endoPDI activity in the desired manner.

The method may comprise contacting isolated endoPDI with the candidate substance in a cell-free assay.

Alternatively the method may comprise contacting a cell expressing endoPDI with the candidate substance.

The cell may be any cell capable of expressing endoPDI under suitable conditions. This includes cells which naturally express endoPDI, (e.g. endothelial cells, multiple myeloma cells) as well as cells engineered to express endoPDI.

The present invention further provides a method for testing the ability of a candidate substance to modulate endoPDI expression, comprising contacting a cell capable of expressing endoPDI with the candidate substance.

The method may further comprise the step of determining the effect of the candidate substance on endoPDI expression. This may be achieved by determining the level of endoPDI protein or mRNA expression. Such determination may be Quantitative or qualitative.

The method may, additionally or alternatively, comprise determining the effect of the candidate substance on endoPDI activity, and/or on the viability of the cell, e.g. determining the presence, absence or amount of one or more markers of apoptosis.

The candidate substance may exert its modulatory effects at any stage of endoPDI expression. Possible mechanisms of action include modulation of transcription (e.g. by affecting binding of transcription factors or RNA polymerase to the endoPDI promoter or other transcriptional regulatory sequences), post-transcriptional RNA processing (e.g. capping, polyadenylation or splicing), turnover of endoPDI RNA within the cell, or translation of mRNA into protein.

The cell may be any cell capable of expressing endoPDI under suitable conditions. This includes cells which naturally express endoPDI, (e.g. endothelial cells, multiple myeloma cells) as well as cells engineered to express endoPDI. Expression of endoPDI may be either constitutive or induced in response to a given stimulus. For example, endothelial cells increase their expression of endoPDI under hypoxic conditions. However certain cell types, such as myeloma cells, appear to express high levels of endoPDI constitutively.

The method may comprise causing the candidate substance to be expressed by the cell. The candidate substance may be a nucleic acid capable of binding to endoPDI mRNA, which may be referred to as an anti-sense agent (e.g. antisense RNA, including siRNA, or a ribozyme). Such agents may prevent translation of the RNA, or may trigger degradation of the mRNA by the cell, or may directly cleave the mRNA. Alternatively the candidate substance may be a protein which may act e.g. to inhibit transcription of the gene or translation of the mRNA. In such methods the cell typically comprises DNA encoding the candidate substance, operably linked to a promoter and/or other regulatory elements providing appropriate transcriptional control, e.g. as part of an expression vector.

The method may further comprise the step of determining the effect of the candidate substance on expression of one or more control proteins as described above. This may be assessed in the same cell in which the effect on endoPDI expression is determined, or in a different cell.

Modulators of endoPDI transcription may also be identified by means of an assay in which a reporter gene (normally comprising a coding sequence other than for endoPDI itself) is coupled to the endoPDI transcriptional control sequences (e.g the promoter). Thus the present invention also provides a method for testing the ability of a candidate substance to modulate endoPDI expression, comprising contacting a cell with the candidate substance, the cell comprising nucleic acid encoding a reporter operably linked to an endoPDI transcriptional regulatory sequence.

The present invention further provides endoPDI modulators identified by the methods described herein. In particular the invention provides endoPDI antagonists identified by the methods described herein.

Further aspects of the present invention derive from the finding that endoPDI is specifically expressed in endothelial cells under conditions of hypoxia, and that endoPDI is required for survival of endothelial cells under hypoxic, but not normoxic, conditions.

Furthermore, archetypal human PDI has been identified at the cell surface, despite possessing a KDEL motif which normally results in localisation in the endoplasmic reticulum. It is thought that endoPDI protein may also be expressed at the cell surface.

Thus endoPDI may serve as both a marker for angiogenesis and a possible therapeutic target for treatment of conditions characterised by angiogenesis. Agents capable of binding to endoPDI RNA or protein may be useful in labelling of cells expressing endoPDI for diagnostic purposes. They may also be useful in targeting therapeutic agents to sites of angiogenesis.

Therefore the present invention provides a method of labelling an endothelial cell comprising contacting said cell with a binding agent capable of detecting expression of endoPDI. The endothelial cell may be experiencing hypoxic conditions.

The binding agent may be capable of binding to endoPDI protein (e.g. it may be an antibody), or it may be capable of binding specifically to endoPDI mRNA (e.g. a nucleic acid molecule, such as a DNA or RNA probe, complementary to a portion of endoPDI mRNA).

Such a method is particularly useful for labelling an endothelial cell at a site of angiogenesis, especially angiogenesis stimulated by hypoxia. The method may be performed in vitro on a biological sample extracted from a subject. The method may be performed on whole or fixed cells, e.g. in a tissue section, or may be an extract or homogenate derived from the sample. Alternatively the method may be performed in vivo, using a binding agent suitable for use in a diagnostic imaging technique, preferably a non-invasive technique such as X-ray, magnetic resonance imaging, etc. For example, a binding agent capable of binding specifically to endoPDI may be used to detect sites of angiogenesis in a subject, for example at sites of solid tumours.

When performed in vitro or in vivo, the binding agent may be labelled to allow visualisation or other suitable detection of its binding to the endothelial cell. Alternatively the method may comprise the further step of contacting the cell with a developing agent. This developing agent is typically capable of binding specifically to the first (or primary) binding agent.

The invention further provides a method of detecting angiogenesis, comprising contacting a biological sample with a binding agent for endoPDI, determining the presence, absence or amount of endoPDI expression and optionally correlating the result with occurence of angiogenesis, or a condition associated with angiogenesis such as a solid tumour. The method may involve comparing the result with a result obtained from a normal sample, e.g. a sample from a healthy individual.

The invention further provides methods of treating conditions characterised by angiogenesis. The methods may comprise administration of an endoPDI antagonist, whereby the endoPDI antagonist inhibits angiogenesis by reducing endoPDI activity and/or expression. The methods may comprise administration of a binding agent capable of binding to endoPDI. The binding agent may itself be an endoPDI antagonist. Alternatively a therapeutic agent may be targeted to a site of angiogenesis via the binding agent.

The binding agent may bind to endoPDI RNA (e.g. an antisense agent), but preferably binds endoPDI protein.

In particular endoPDI antagonists and binding agents may be used for the treatment of tumours, and particularly for the treatment of solid tumours, although other conditions characterised by angiogenesis may be treated by such agents (see below). Growth of solid tumours is often characterised by angiogenesis triggered by the hypoxic environment within the tumour cell mass. Thus an endoPDI antagonist may be used to inhibit angiogenesis at the tumour site. An endoPDI binding agent may be used to target a therapeutic agent to the site of tumour growth. The therapeutic agent may inhibit angiogenesis, or may instead act directly on the tumour.

The invention thus provides an endoPDI binding agent for use in a method of medical treatment.

The invention further provides an endoPDI binding agent for use in the inhibition of angiogenesis, e.g. in the treatment of a condition characterised by angiogenesis. Such conditions include any in which angiogenesis contributes to the pathology and which could therefore be treated in whole or in part by inhibition of angiogenesis. Examples include psoriasis, diabetic retinopathy, endometriosis, atherosclerosis, rheumatoid arthritis, Alzheimer's disease and solid tumours.

The invention further provides the use of an endoPDI binding agent in the manufacture of a medicament for use in the inhibition of angiogenesis, e.g. in the treatment of a condition characterised by angiogenesis, such as a solid tumour.

The present inventors have shown that inhibition of endoPDI activity results in death of endothelial cells. Thus in further embodiments the present invention provides a method of inhibiting angiogenesis comprising contacting an endothelial cell with an endoPDI antagonist.

The invention further provides an endoPDI antagonist for use in the inhibition of angiogenesis, e.g. in the treatment of a condition characterised by angiogenesis such as solid tumours and others as described elsewhere herein.

Also provided is the use of an endoPDI antagonist in the preparation of a medicament for the inhibition of angiogenesis. Such a medicament may be useful in the treatment of cancer, in particular in the treatment of a solid tumour, and of other conditions characterised by angiogenesis.

Lead compounds identified by the methods of the invention may be formulated for use as pharmaceuticals, e.g. by formulation with a pharmaceutically acceptable carrier. The invention further provides a method of formulating a pharmaceutical composition comprising, having identified a substance as an endoPDI antagonist, formulating it with a pharmaceutically acceptable carrier.

Lead compounds may also be optimised for pharmaceutical administration. For example, a lead compound may be used as the basis for the design of mimetics having altered (especially improved) characteristics including, but not limited to, ease of synthesis, activity, specificity, pharmaceutical acceptability, half-life in a subject, etc.

The methods and compositions of the present invention are preferably used for the treatment of mammals, and in particular for the treatment of humans, other primates (including great apes and Old and New World monkeys), livestock (including horses, cows, pigs, etc.), rodents (including mice and rats), and household pets and common laboratory animals (including cats, dogs, guinea pigs and rabbits).

The present invention further provides methods of screening for substances useful in the inhibition of angiogenesis.

Thus the present invention provides a method for assessing the ability of a candidate substance to inhibit angiogenesis, comprising contacting an endothelial cell with the candidate substance. The method may further comprise assessing the effect of the candidate substance on endoPDI activity or expression. Additionally or alternatively the candidate substance may previously have been identified as an endoPDI antagonist by the methods described herein.

The endothelial cell is preferably hypoxic. Preferably the method comprises further contacting a normoxic endothelial cell with the candidate substance

The method preferably comprises assessing whether or not the endothelial cell undergoes apoptosis.

The method may comprise causing the candidate substance to be expressed by the cell or cells.

The present invention further provides an expression vector comprising nucleic acid encoding a protein having endoPDI activity operably linked to a promoter. Also provided is a host cell comprising an expression vector as described herein.

The present invention further provides an isolated protein having endoPDI activity. By isolated is meant separated from one or more components with which it is found associated in nature, e.g. separated from one or more components of a cell in which it is expressed. Also provided is an isolated protein with protein disulphide isomerase activity having at least 80% identity, and preferably 85, 90, 95 or 100% identity with the published sequences described below.

The endoPDI promoter may be used to direct expression of a desired substance, encoded by a nucleic acid sequence, in a target cell. Thus the present invention provides a method of controlling expression of a desired substance in a target cell, comprising introducing a nucleic acid expression construct into the target cell, the construct comprising the a nucleic acid sequence encoding the desired substance operably linked to the endoPDI promoter, whereby the endoPDI promoter drives expression of the desired nucleic acid sequence.

In preferred embodiments the target cell is an endothelial cell. Preferably expression of the desired nucleic acid sequence is increased by hypoxic conditions.

The invention further provides an isolated nucleic acid molecule or construct comprising a desired nucleic acid sequence operably linked to the endoPDI promoter. Also provided is a vector comprising said nucleic acid molecule, and a host cell comprising said nucleic acid molecule or said vector. Preferably the desired nucleic acid sequence is other than the endoPDI coding sequence. Preferably the nucleic acid sequence encodes a reporter gene, an antisense agent (i.e. a nucleic acid capable of binding to endoPDI mRNA), or an enzyme capable of converting a prodrug to an active form.

### BRIEF DESCRIPTION OF THE DRAWINGS

Table 1. Expression of EndoPDI in SAGE libraries of normal tissues.

The NCBI database for serial analysis of gene expression (SAGE) was used to examine the relative expression of EndoPDI. EndoPDI expression was found in a total of 211 libraries, and of these, 26 were derived from normal tissues. The tags per million counts for EndoPDI in these 26 libraries is shown in the table.

FIG. 1. Sequence homology and structural organisation of EndoPDI.

A: Homology alignment of human EndoPDI with other species. Comparison of the human EndoPDI sequence with genome databases for other species identified homologues of EndoPDI in the rat, mouse, Xenopus, Drosophila and mosquito.

B: Structural comparison of EndoPDI with other members of the PDI family. In the diagram, PDIs are classified according to the presence of the PDI CXXC or Trx motif, together with the KDEL endoplasmic reticulum retention sequence. The domains containing the CXXC motif are also known as ‘a’ domains and the domains containing no active site but containing the thioredoxin structural fold are known as ‘b’ domains. Unlike, the other members of the family, EndoPDI appears to contain no b domain, rather it has the structural organisation ao, a, a′, c.

FIG. 2. RNase protection analysis of EndoPDI expression by cell lines in vitro.

The level of EndoPDI mRNA expression was determined in 10 different cell types; MRC-5 (fibroblast), SY—SH—SY (neuroblastoma), SK23 (skin fibroblast), MDA468 (breast carcinoma), NCIM520 (squamous cell lung carcinoma), ZR75 (oestrogen dependent breast carcinoma), HL60 (promyelocytic leukemia), HMME (immortalised endothelial cell line), HUVEC (human umbilical vein endothelial cells) and HDMEC (human dermal microvascular endothelial cells) by RNase protection assay. Quantitation was performed using a phosphorimager and quantitation software. The relative abundance of EndoPDI in each cell type tested is shown in the bar chart.

FIG. 3. Multiple tissue array analysis of EndoPDI expression.

A: Relative abundance of EndoPDI mRNA in normal tissues. Normal tissue blots were used to determine the level of EndoPDI expression in human tissues. The bar chart shows the relative expression above background for each tissue. The tissues have been grouped into cardiac, gastro-intestinal and others.

B: Relative abundance of EndoPDI in matched normal versus tumour tissue. Matched tumour and normal tissue blots were used to examine the relative expression of EndoPDI in tumour and normal tissues. The number of patient samples in each group were as follows; cervix n=1, uterus n=3, stomach n=8, lung n=6.

FIG. 4. Demonstration of EndoPDI in human tissues by in situ hybridisation.

Moderate expression (arrows) of EndoPDI is seen in the endothelium of a melanoma (A and C bright field; B and D dark field). High expression (arrows) is seen in the syncyntiotrophoblast cells of placenta (E bright field; F dark field). There is a detectable signal (arrow) in the endothelial cells and macrophages of an atherosclerotic plaque (G bright field; H dark field). Very strong signal was detected in the keratinocytes of a human skin hair follicle (I bright field; J dark field).

FIG. 5. Induction of EndoPDI expression by hypoxia.

RNase protection and Western blotting analysis were used to measure the expression of EndoPDI mRNA (A) and protein (B) after 1, 4, 8, 16 and 24 h of exposure to 0.1% oxygen. Induction of EndoPDI mRNA was seen after 1 h and during 16 h of hypoxia. Induction of EndoPDI protein was seen after 4 h and during at least 24 h of hypoxia.

FIG. 6. Downregulation of EndoPDI mRNA and protein under normoxia and hypoxia using RNA interference.

RNase protection and Western blotting analysis was used to measure the expression of EndoPDI mRNA and protein respectively after treatment with siRNA specific to EndoPDI under normoxia and hypoxia. There was a complete loss of EndoPDI mRNA (A) and protein (B) expression after siRNA treatment under both normoxia and hypoxia.

FIG. 7. EndoPDI protects human microvascular endothelial cells from undergoing apoptosis under hypoxia but not normoxia.

FACS analysis was used to measure apoptosis in human microvascular endothelial cells after transfection with EndoPDI specific siRNA under both normoxia (A) and hypoxia (B). Cells were treated with transfection reagents alone (control), EndoPDI specific siRNA (RNAi) or scrambled siRNA (Scrambled). The percentages of cells in the necrotic (black bars) as well as the apoptotic (grey bars) populations are shown in the bar chart. There was a significant increase in the apoptotic population compared with controls under hypoxia (0.1% O2, 16 h) but not normoxia after EndoPDI downregulation by siRNA. The results are the means of 3 replicate experiments ±S.D.

FIG. 8. PDI protects endothelial cells from undergoing apoptosis under both normoxia and hypoxia. FACS analysis was used to measure apoptosis in HDMEC after treatment with PDI specific siRNA. There was a significant increase in the apoptotic population compared with controls in both normoxia and hypoxia treated cells (A). Panel B shows the comparison with EndoPDI specific siRNA treated cells. Both sets of data were normalised to controls. The results are the means of 3 replicate experiments ±S.D.

FIG. 9. Effect of EndoPDI down-regulation by siRNA on the expression of adrenomedullin, endothelin-1 and CD105 under hypoxia. The secretion of adrenomedullin by HDMEC (A) was measured by radioimmunoassay after down-regulation of PDI, EndoPDI or both PDI and EndoPDI under hypoxia. The secretion of endothelin-1 by HDMEC was measured by ELISA (B) and the cell surface expression of CD105 (C) by FACS following the same treatment. In panel C for each histogram, the dark solid line represents CD105 cell surface expression under hypoxia and the lighter solid line represents CD105 expression under normoxia. The dotted line for the top histogram represents CD105 cell surface expression after treatment with PDI specific siRNA, after treatment with EndoPDI specific siRNA for the middle histogram and after treatment with siRNA to both PDI and EndoPDI for the bottom histogram.

FIG. 10. Sequences of human endoPDI. (A) The human endoPDI cDNA sequence; (B) the predicted amino acid sequence of the full length protein.

### DETAILED DESCRIPTION OF THE INVENTION

EndoPDI

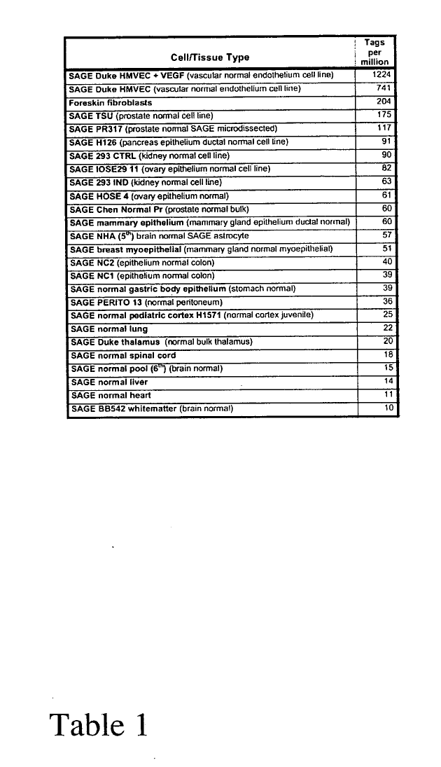

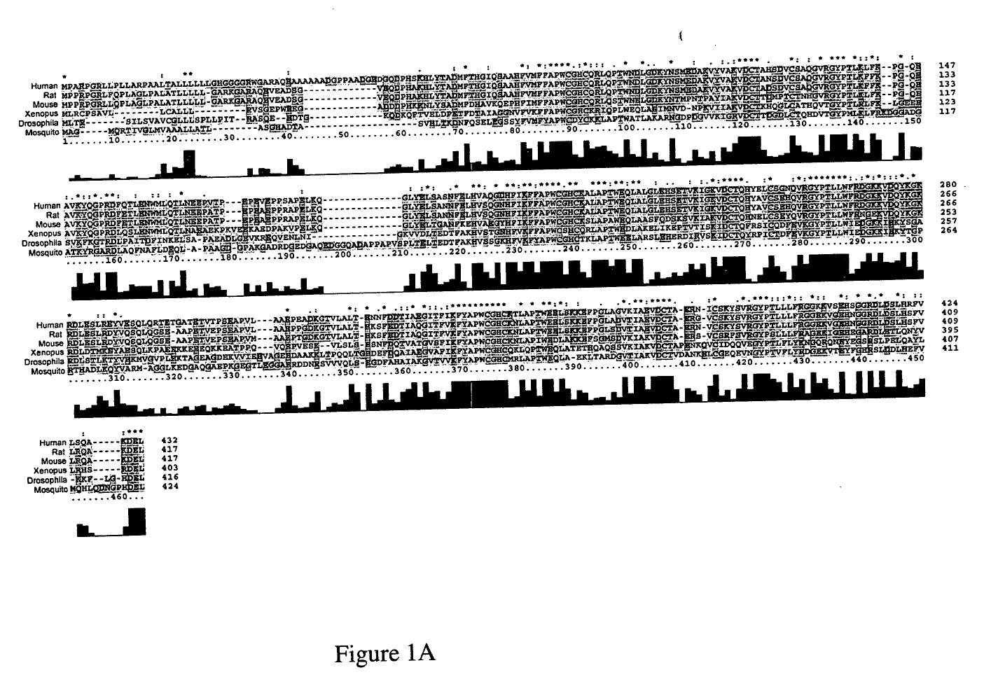

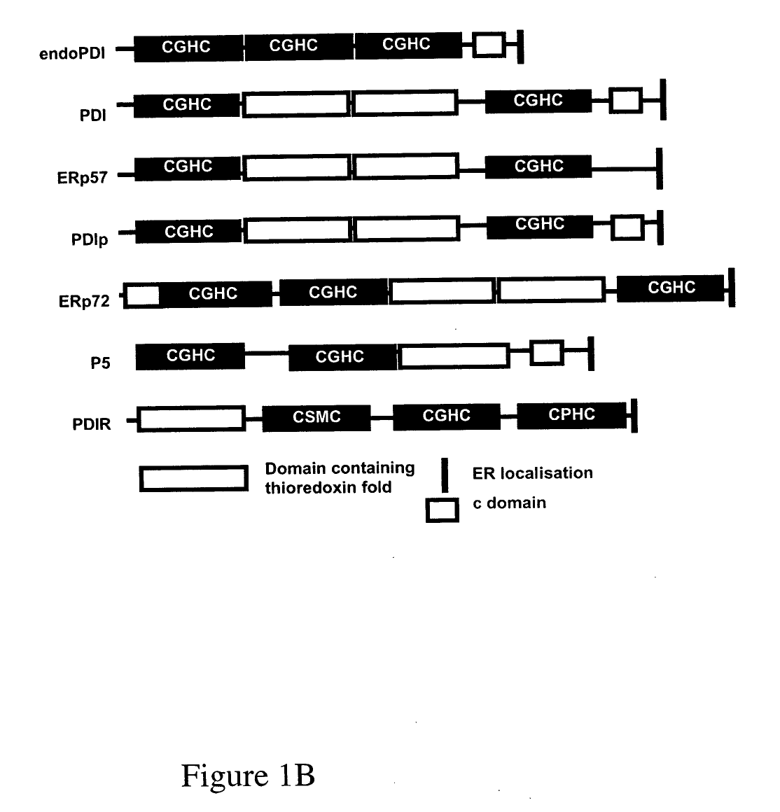

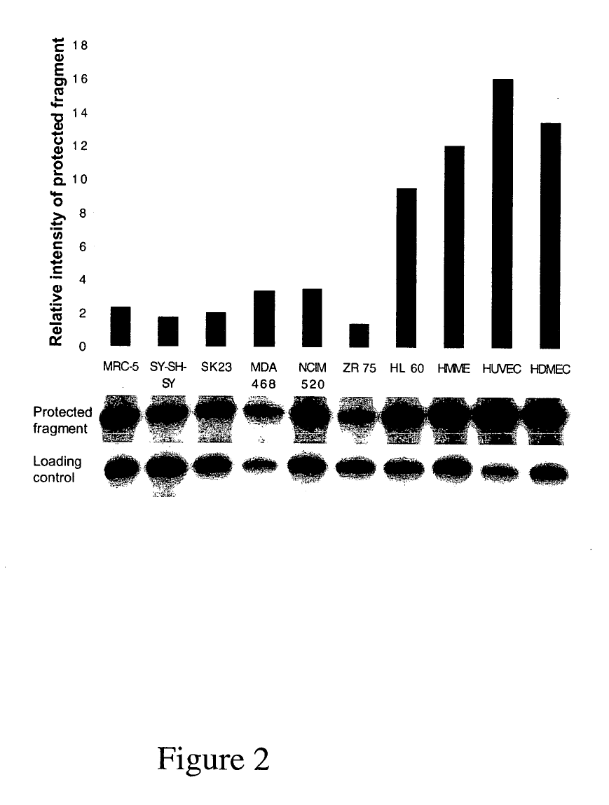

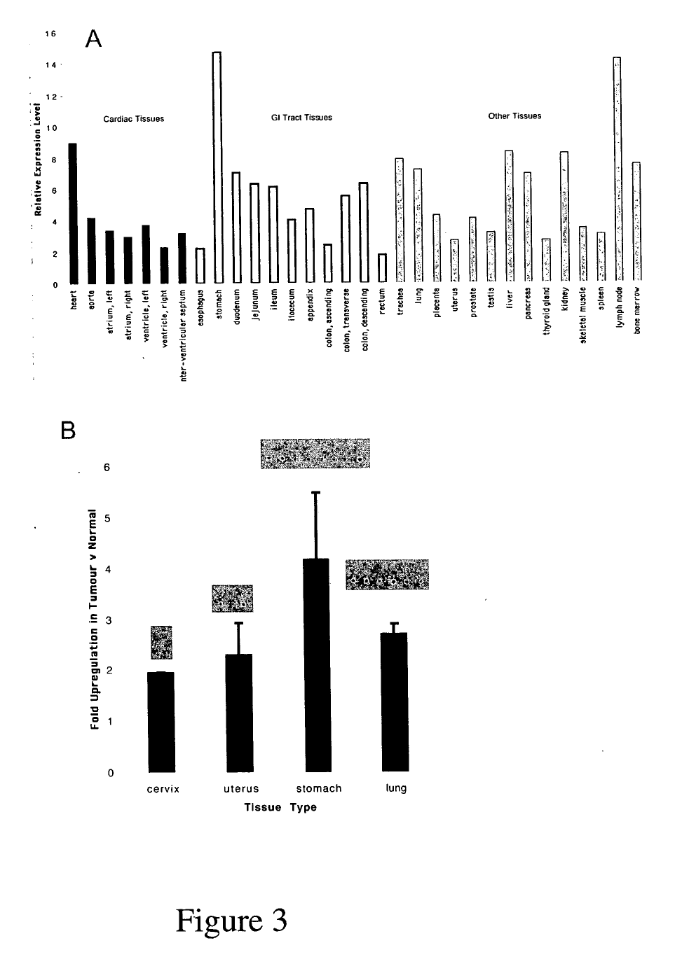

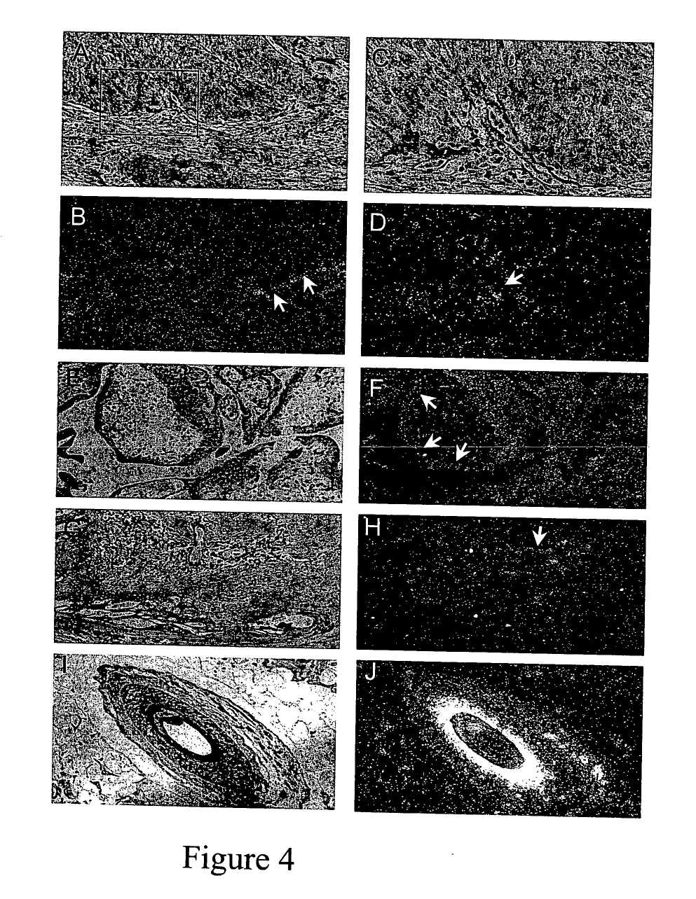

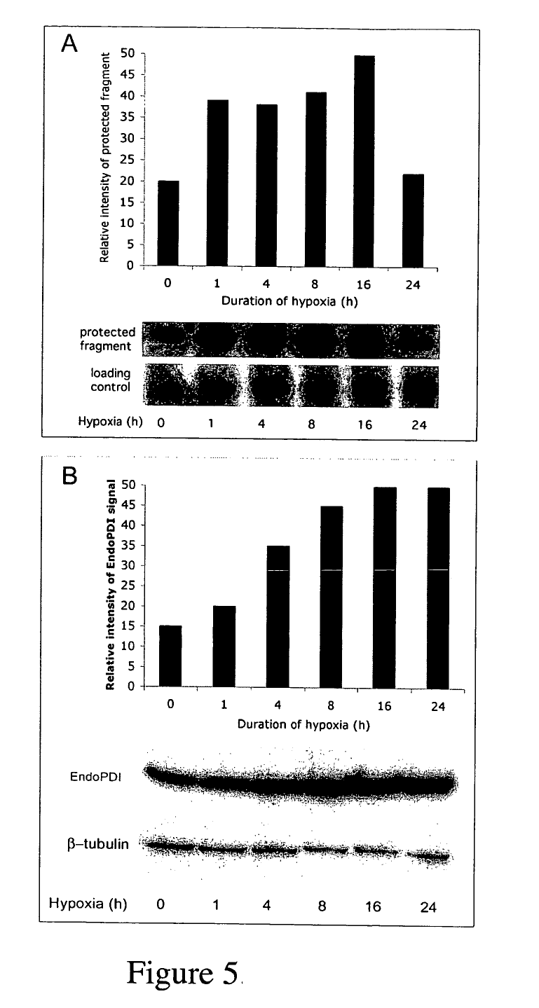

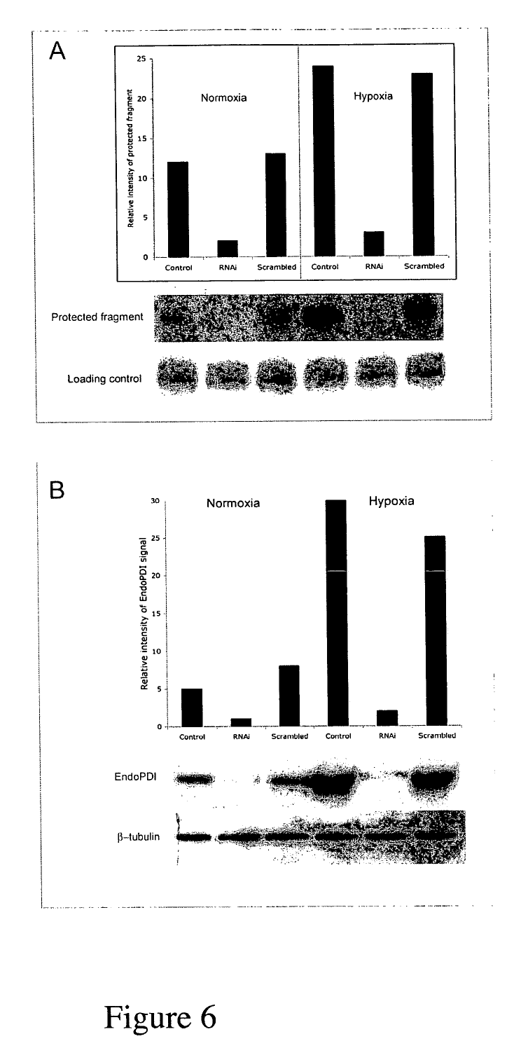

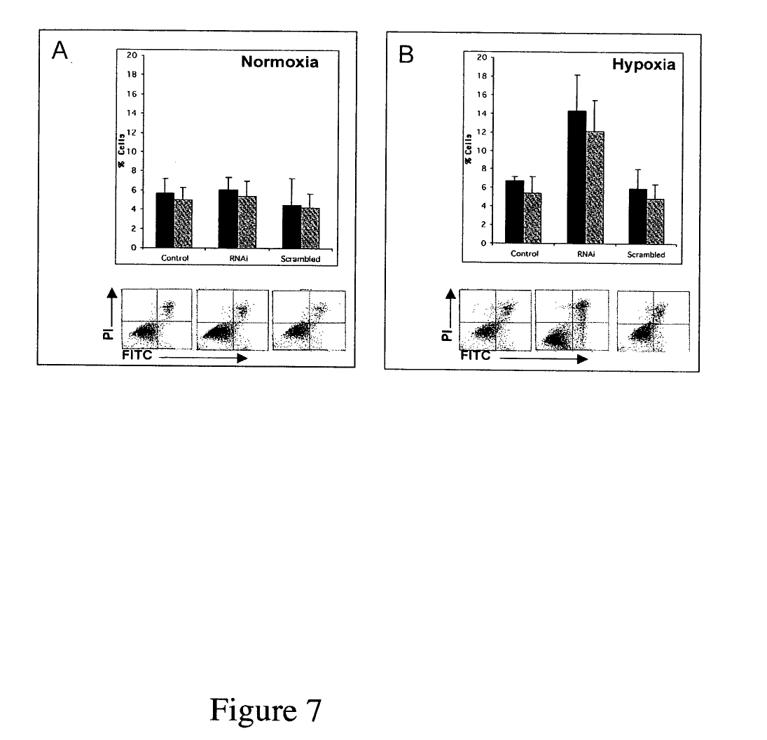

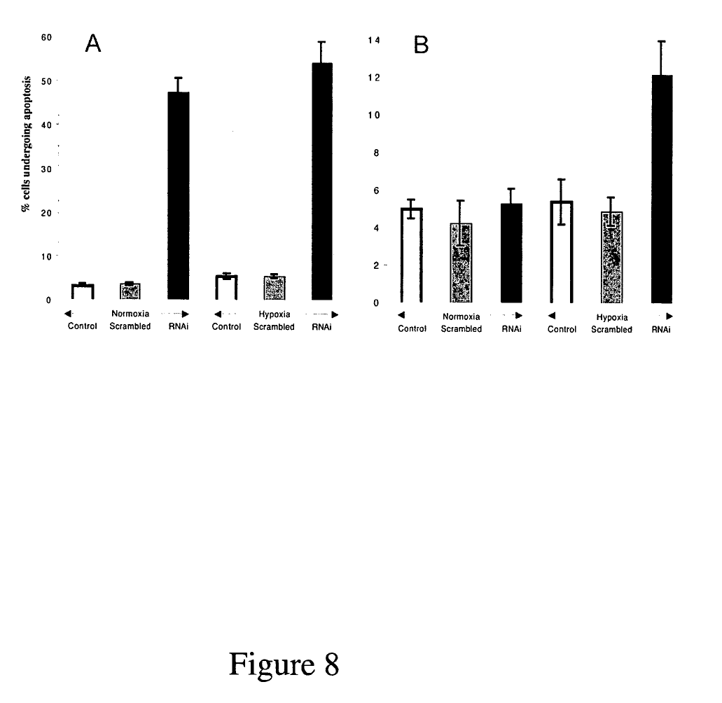

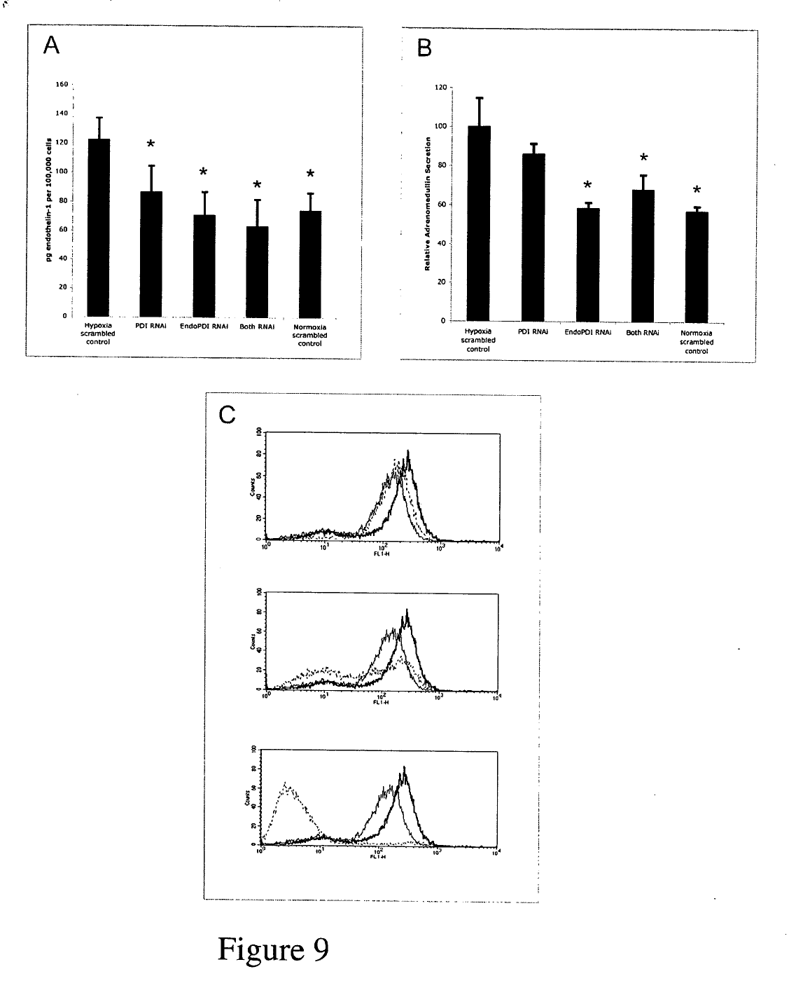

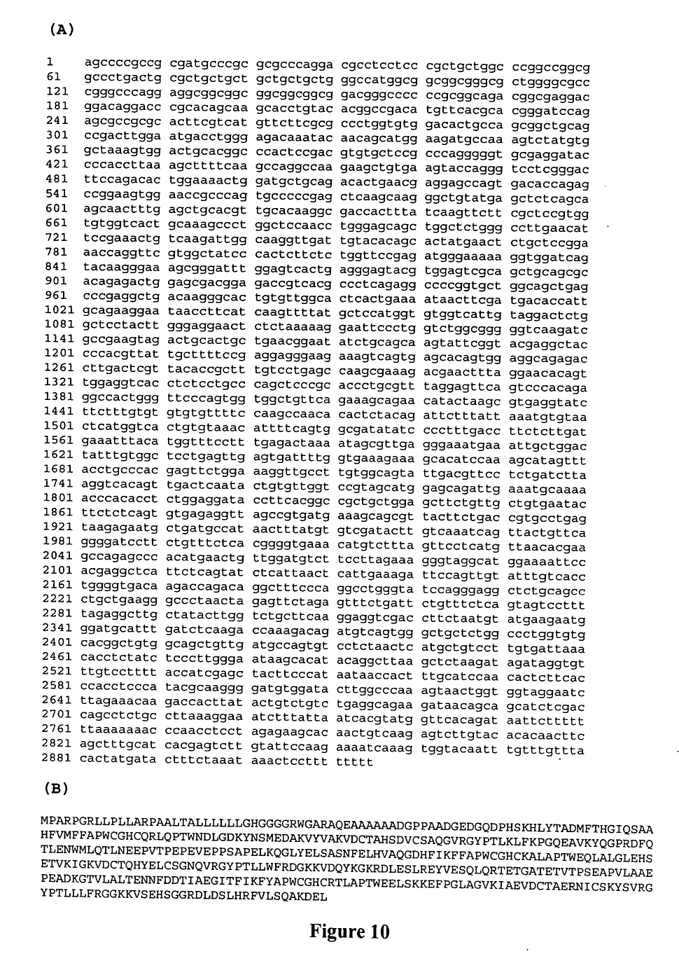

EndoPDI proteins include proteins having the amino acid sequence shown in GenBank entry AK075291 and as shown on the top line of the alignment in FIG. 1A, and to proteins having at least 75% identity to it, preferably at least 80%, identity, preferably at least 85% identity, preferably at least 90%, 91%, 92%, 93%, 94%, 95%, 96%, 97%, 98% or 99% sequence identity to that sequence. Percentage sequence identity may be calculated using a program such as BLAST or BestFit from within the Genetics Computer Group (GCG) Version 10 software package available from the University of Wisconsin, using default parameters.

The protein preferably has disulfide isomerase activity, that is to say it is capable of catalysing at least the cleavage or formation of a disulphide bond in a suitable substrate, and preferably is capable of catalysing both cleavage and formation (rearrangement) of disulphide bonds in a suitable substrate. However proteins lacking one or more of these activities may also fall within the definition of endoPDI proteins.

EndoPDI nucleic acids include any nucleic acid encoding an endoPDI protein as defined above. This includes the native human coding sequence (GenBank accession number AK075291; GI:22761284) and variants, derivatives and mutants thereof, including orthologous coding sequences in other species and naturally occurring allelic variants, as well as man-made or other mutants. Orthologous sequences are homologous sequences in different organisms which are derived from a common ancestral precursor. EndoPDI sequences have been identified in a number of species (see FIG. 1). Preferred species for the purposes of the present invention are mammalian species, including humans, other primates (including great apes and Old and New World monkeys), rodents (including mice and rats) and other common laboratory animals (such as rabbits, guinea pigs etc).

EndoPDI nucleic acids include any nucleic acid sequence having at least 75% identity to the coding sequence of the human clone AK075291 (GI:22761284) over a stretch of at least 100, 200, 300, 400, 500, or 1000 contiguous nucleotides, preferably at least 80%, identity, preferably at least 85% identity, preferably at least 90%, 91%, 92%, 93%, 94%, 95%, 96%, 97%, 98% or 99% sequence identity to that sequence. Percentage sequence identity may be calculated using a program such as BLAST or BestFit from within the Genetics Computer Group (GCG) Version 10 software package available from the University of Wisconsin, using default parameters.

EndoPDI nucleic acids may also comprise non-coding regions, including 5′ and 3′ untranslated regions of the mature mRNA, as well as the transcriptional regulatory sequences, including the promoter (see below).

Nucleic acids having the appropriate level of sequence homology with the coding region of the published human sequence (AK075291; GI:22761284) may be identified by using hybridization and washing conditions of appropriate stringency. For example, hybridizations may be performed, according to the method of Sambrook et al., (“Molecular Cloning, A Laboratory Manual, Cold Spring Harbor Laboratory Press, 1989) using a hybridization solution comprising: 5×SSC, 5× Denhardt's reagent, 0.5-1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

One common formula for calculating the stringency conditions required to achieve hybridization between nucleic acid molecules of a specified sequence homology is (Sambrook et al., 1989):
Tm=81.5° C.+16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tm is 57° C. The Tm of a DNA duplex decreases by 1-1.5° C. with every 1% decrease in homology. Thus, targets with greater than about 75% sequence identity would be observed using a hybridization temperature of 42° C. Such a sequence would be considered substantially homologous to the endoPDI nucleic acid sequence.

Nucleic acids according to the present invention may be provided isolated and/or purified from their natural environment, in substantially pure or homogeneous form, or free or substantially free of other nucleic acids of the species of origin. Where used herein, the term “isolated” encompasses all of these possibilities.

The nucleic acids may be wholly or partially synthetic. In particular they may be recombinant in that nucleic acid sequences which are not found together in nature (do not run contiguously) have been ligated or otherwise combined artificially. Alternatively they may have been synthesised directly e.g. using an automated synthesiser.

Nucleic acid according to the present invention may be polynucleotides or oligonucleotides, and may include cDNA, RNA, genomic DNA and modified nucleic acids or nucleic acid analogs. Where a DNA sequence is specified, e.g. with reference to a figure, unless context requires otherwise the RNA equivalent, with U substituted for T where it occurs, is encompassed.

Nucleic acids may comprise, consist or consist essentially of any of the sequences disclosed herein (which may be a gene, a genomic clone or other sequence, a cDNA, or an ORF or exon of any of these etc.). For example, where gDNA is disclosed, nucleic acids comprising any one or more introns or exons from any of the gDNA are also embraced. Likewise, where cDNA is disclosed, nucleic acids comprising only the translated region (from initiation to termination codons) are also embraced.

Where a nucleic acid (or nucleotide sequence) of the invention is referred to herein, the complement of that nucleic acid (or nucleotide sequence) will also be embraced by the invention. The ‘complement’ in each case is the same length as the reference, but is 100% complementary thereto whereby by each nucleotide is base paired to its counterpart i.e. G to C, and A to T or U.

Protein Disulphide Isomerases

Protein disulphide isomerases catalyse the exchange of cysteine partners in disulphide bonds. This exchange reaction typically involves reduction of one or more disulphide bond in a target molecule followed by oxidation of alternative cysteine pairs to form the required disulphide pair(s). Under appropriate assay conditions, this exchange reaction can be driven to either formation or destruction of disulphide bonds.

Protein disulphide isomerases include archetypal human PDI (NM_{—}000918.2 GI:20070124), ERp57 (NM_{—}005313.3 GI:21361656), PDIp (NM_{—}006849.1 GI:5803118), P5 (BC001312.1 GI:12654930), ERp72 (J05016.1 GI:181507) and PDIR (NM_{—}006810.1 GI:5803120).

Assays for protein disulphide isomerase enzymatic activity typically exploit the capacity of the enzyme to catalyse the cleavage and/or formation of a disulphide bond in a suitable substrate.

Examples of possible assay methods are set out below. However the skilled person will be perfectly capable of adapting these assays or designing alternatives depending on their particular requirements.

Restoration of activity to an unfolded or misfolded protein.

An unfolded or misfolded protein (generated e.g. by rapid heating and cooling) may be contacted with anzyme, and restoration of its activity followed by any suitable means. Spectroscopic methods, involving measurement of e.g. fluorescence, absorbance or luminescence may be particularly convenient. The protein may be an enzyme capable of acting on a substrate to produce a spectrophotometrically detectable change. Nucleases such as RNase or DNase are particularly preferred. For example, refolding of RNAse is a well known laboratory based assay for PDI. The activity of refolded RNAse can be detected by its activity to degrade a fluorescently labelled RNA reporter molecule. The degradation of the labelled reporter RNA can be measured by decrease in Fluorescent Polarisation (FP) as degradation proceeds.

Direct addition of a labelled substrate to a carrier. Numerous variations of this type of assay are possible. Either or both of the substrate and carrier may be labelled. Selected examples follow.

A radiolabelled molecule such as glutathione or a phosphopeptide may be coupled to cysteine derivatised scintillation media.

A fluorescently labelled cysteine-containing substrate may be coupled to a carrier, and the change in fluorescence polarisation of the substrate monitored accordingly. For example, a small fluorescently labelled peptide containing cysteine can be reacted with a larger carrier protein thereby increasing its size and therefore its FP

A fluorescently labelled cysteine-containing substrate may be coupled to a carrier which also carries a fluorescent label. The coupling reaction may be followed by fluorescence energy resonance transfer between the two labels.

Depolymerisation of a dimeric or multimeric substrate having monomers linked by disulphide bonds. The depolymerisation reaction may be observed through the increase in concentration of free monomer, or reduction in size of the polymer. The reaction may be driven towards depolymerisation by allowing free monomer to react with an inert carrier (e.g. glutathione) in order for it not to re-enter the reaction cycle and recombine with the multimer. Depolymerisation may be followed by FRET, fluorescence, absorbance, or fluorescence polarisation.

Disruption or intermolecular exchange of one or more disulphide bonds in a suitably labelled reporter molecule.

The reporter molecule will typically be a peptide or protein. It may be labelled, for example, with a combination of a fluorescent label and a quenching moiety, wherein the quenching moiety reduces or abrogates fluorescence of the label. Upon disruption of the disulphide bond(s) the quenching moiety and fluorescent label are separated in space, reducing or removing the quenching effect and giving a measurable signal. This signal could be stabilised by the addition of excess inert free cysteine containing molecules to disfavour rejoining of the intermolecular disulphide Such a reporter molecule may be referred to as a peptide beacon.

Hypoxia

Hypoxia is an oxygen concentration less than that present in normal tissues, i.e. less than about 4%.

Inhibition of EndoPDI Expression

Expression of endoPDI may be modulated, and preferably down-regulated or completely abrogated, by methods based on the use of molecules, preferably RNA molecules, capable of hybridising to the endoPDI transcript. These molecules are referred to as anti-sense agents, and include anti-sense RNA, siRNA, ribozymes, etc.

Thus included within the scope of the invention are antisense oligonucleotide sequences based on the endoPDI nucleic acid sequence. These may themselves be useful in a therapeutic context, e.g. they may be designed to hybridize to the complementary sequence of nucleic acid, pre-mRNA or mature mRNA, interfering with the production of native endoPDI polypeptide, so that its expression is reduced or prevented altogether. The construction of antisense sequences and their use is described in Peyman and Ulman, Chemical Reviews, 90:543-584, (1990), Crooke, Ann. Rev. Pharmacol. Toxicol., 32:329-376, (1992), and Zamecnik and Stephenson, P.N.A.S, 75:280-284, (1974).

In using anti-sense agents, including genes or partial gene sequences, to down-regulate gene expression, a nucleotide sequence is placed under the control of a promoter in a “reverse orientation” such that transcription yields RNA which is complementary to normal mRNA transcribed from the “sense” strand of the target gene.

Thus a nucleotide sequence which is complementary to an endoPDI nucleic acid sequence, and particularly a coding sequence, forms one part of the present invention.

Further options for down regulation of gene expression include the use of ribozymes, e.g. hammerhead ribozymes, which can catalyse the site-specific cleavage of RNA, such as mRNA (see e.g. Jaeger (1997) “The new world of ribozymes” Curr. Opin. Struct. Biol. 7:324-335, or Gibson & Shillitoe (1997) “Ribozymes: their functions and strategies for their use” Mol. Biotechnol. 7: 242-251.)

The complete sequence corresponding to the coding sequence (in reverse orientation for anti-sense) need not be used. For example fragments of sufficient length may be used. It is a routine matter for the person skilled in the art to screen fragments of various sizes and from various parts of the coding sequence to optimise the level of anti-sense inhibition. It may be advantageous to include the initiating methionine ATG codon, and perhaps one or more nucleotides upstream of the initiating codon. A further possibility is to target a conserved sequence of a gene, e.g. a sequence that is characteristic of one or more genes, such as a regulatory sequence.

The sequence employed may be about 500 nucleotides or less, possibly about 400 nucleotides, about 300 nucleotides, about 200 nucleotides, or about 100 nucleotides. It may be possible to use oligonucleotides of much shorter lengths, e.g. 14, 15, 16, 17, 18, 19, 20, 21, 22, 23, 24 or 25 nucleotides, although longer fragments, and generally even longer than about 500 nucleotides are preferable where possible, such as longer than about 600 nucleotides, than about 700 nucleotides, than about 800 nucleotides, than about 1000 nucleotides or more.

It may be preferable that there is complete sequence identity in the sequence used for down-regulation of expression of a target sequence, and the target sequence, although total complementarity or similarity of sequence is not essential. One or more nucleotides may differ in the sequence used from the target gene. Thus, a sequence employed in a down-regulation of gene expression in accordance with the present invention may be a wild-type sequence (e.g. gene) selected from those available, or a variant of such a sequence.

The sequence need not include an open reading frame or specify an RNA that would be translatable. It may be preferred for there to be sufficient homology for the respective anti-sense and sense RNA molecules to hybridise. There may be down regulation of gene expression even where there is about 5%, 10%, 15% or 20% or more mismatch between the sequence used and the target gene. Effectively, the homology should be sufficient for the down-regulation of gene expression to take place.

Thus the present invention further provides the use of an endoPDI nucleotide sequence, or its complement, or a variant of either for down-regulation of endoPDI gene expression. This may be useful in a therapeutic context, in particular to influence the growth or survival of an endothelial cell, especially under hypoxic conditions.

Thus, the present invention also provides a method of influencing, preferably suppressing, angiogenesis, the method including causing or allowing expression from nucleic acid according to the invention within endothelial cells.

Anti-sense or sense regulation may itself be regulated by employing an inducible promoter in an appropriate construct.

Double stranded RNA (dsRNA) has been found to be even more effective in gene silencing than both sense or antisense strands alone (Fire A. et al Nature, Vol 391, (1998)). dsRNA mediated silencing is gene specific and is often termed RNA interference (RNAi) (See also Fire (1999) Trends Genet. 15: 358-363, Sharp (2001) Genes Dev. 15: 485-490, Hammond et al. (2001) Nature Rev. Genes 2: 1110-1119 and Tuschl (2001) Chem. Biochem. 2: 239-245).

RNA interference is a two step process. First, dsRNA is cleaved within the cell to yield short interfering RNAs (siRNAs) of about 21-23 nt length with 5′ terminal phosphate and 3′ short overhangs (˜2 nt) The siRNAs target the corresponding mRNA sequence specifically for destruction (Zamore P. D. Nature Structural Biology, 8, 9, 746-750, (2001)

Thus in one embodiment, the invention provides double stranded RNA comprising an endoPDI-encoding sequence, which may for example be a “long” double stranded RNA (which will be processed to siRNA, e.g., as described above). These RNA products may be synthesised in vitro, e.g., by conventional chemical synthesis methods.

RNAi may be also be efficiently induced using chemically synthesized siRNA duplexes of the same structure with 3′-overhang ends (Zamore P D et al Cell, 101, 25-33, (2000)). Synthetic siRNA duplexes have been shown to specifically suppress expression of endogenous and heterologeous genes in a wide range of mammalian cell lines (Elbashir S M. et al. Nature, 411, 494-498, (2001)).

Thus siRNA duplexes containing between 20 and 25 bps, more preferably between 21 and 23 bps, of the endoPDI sequence form one aspect of the invention e.g. as produced synthetically, optionally in protected form to prevent degradation.

Alternatively siRNA may be produced from a vector, in vitro (for recovery and use) or in vivo.

Accordingly, the vector may comprise a nucleic acid sequence encoding endoPDI (including a nucleic acid sequence encoding a variant or fragment thereof), suitable for introducing an siRNA into the cell in any of the ways known in the art, for example, as described in any of references cited herein, which references are specifically incorporated herein by reference.

In one embodiment, the vector may comprise a nucleic acid sequence according to the invention in both the sense and antisense orientation, such that when expressed as RNA the sense and antisense sections will associate to form a double stranded RNA. This may for example be a long double stranded RNA (e.g., more than 23 nts) which may be processed in the cell to produce siRNAs (see for example Myers (2003) Nature Biotechnology 21:324-328).

Alternatively, the double stranded RNA may directly encode the sequences which form the siRNA duplex, as described above. In another embodiment, the sense and antisense sequences are provided on different vectors.

These vectors and RNA products may be useful for example to inhibit de novo production of the endoPDI polypeptide in a cell. They may be used analogously to the expression vectors in the various embodiments of the invention discussed herein.

Diagnostic Methods

Binding agents may be used to detect the presence of endoPDI mRNA or protein in biological samples. Such binding agents include nucleic acids complementary to endoPDI mRNA as described above, and binding agents capable of binding, preferably specifically, to endoPDI protein. These agents may be used to detect endoPDI in samples taken from a subject to detect angiogenesis e.g. at the site of a solid tumour. Such assays are typically performed in vitro, and may involve visualisation of protein or RNA expression in a sample of whole or fixed cells, e.g. by immunocytochemistry, in situ hybridisation or in situ PCR. Alternatively they may be used to detect nucleic acid or protein in a tissue homogenate or cell lysate.

The term “specific binding pair” may be used to describe a pair of molecules comprising a specific binding member (sbm) and a binding partner (bp) therefor which have particular specificity for each other and which in normal conditions bind to each other in preference to binding to other molecules. Examples of specific binding pairs are antigens and antibodies, hormones and receptors and complementary nucleotide sequences. The skilled person will be able to think of many other examples and they do not need to be listed here. Further, the term “specific binding pair” is also applicable where either or both of the specific binding member and binding partner comprise just the binding part of a larger molecule. Thus in the context of antibodies, a specific binding member may comprise just a domain of an antibody (antibody binding domain) which is able to bind to either an epitope of an antigen or a short sequence which although unique to or characteristic of an antigen, is unable to stimulate an antibody response except when conjugated to a carrier protein.

Thus in the context of the present invention endoPDI protein and an antibody specific therefor may be regarded as a specific binding pair.

It is possible to take monoclonal antibodies and use the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB 2188638A or EP-A-239400. A hybridoma producing a monoclonal antibody may be subject to genetic mutation or other changes, which may or may not alter the binding specificity of antibodies produced.

As antibodies can be modified in a number of ways, the term “antibody” should be construed as covering any specific binding substance having an binding domain with the required specificity. Thus, this term covers antibody fragments, derivatives, functional equivalents and homologues of antibodies, including any polypeptide comprising an immunoglobulin binding domain, whether natural or synthetic. Chimaeric molecules comprising an immunoglobulin binding domain, or equivalent, fused to another polypeptide are therefore included. Cloning and expression of chimaeric antibodies are described in EP-A-0120694 and EP-A-0125023.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies”, multivalent or multispecific fragments constructed by gene fusion (WO94/13804; P. Holliger et al Proc. Natl. Acad. Sci. USA 90 6444-6448, 1993).

Diabodies are multimers of polypeptides, each polypeptide comprising a first domain comprising a binding region of an immunoglobulin light chain and a second domain comprising a binding region of an immunoglobulin heavy chain, the two domains being linked (eg by a peptide linker) but unable to associate with each other to form an antigen binding site: antigen binding sites are formed by the association of the first domain of one polypeptide within the multimer with the second domain of another polypeptide within the multimer (WO94/13804).

Where bispecific antibodies are to be used, these may be conventional bispecific antibodies, which can be manufactured in a variety of ways (Holliger, P. and Winter G. Current Opinion Biotechnol. 4, 446-449 (1993)), eg prepared chemically or from hybrid hybridomas, or may be any of the bispecific antibody fragments mentioned above. It may be preferable to use scFv dimers or diabodies rather than whole antibodies. Diabodies and scFv can be constructed without an Fc region, using only variable domains, potentially reducing the effects of anti-idiotypic reaction. Other forms of bispecific antibodies include the single chain “Janusins” described in Traunecker et al, Embo Journal, 10, 3655-3659, (1991).

Bispecific diabodies, as opposed to bispecific whole antibodies, are also particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected.

It may be desirable to “humanise” non-human (eg murine) antibodies to provide antibodies having the antigen binding properties of the non-human antibody, while minimising the immunogenic response of the antibodies, eg when they are used in human therapy. Thus, humanised antibodies comprise framework regions derived from human immunoglobulins (acceptor antibody) in which residues from one or more complementary determining regions (CDR's) are replaced by residues from CDR's of a non-human species (donor antibody) such as mouse, rat or rabbit antibody having the desired properties, eg specificity, affinity or capacity. Some of the framework residues of the human antibody may also be replaced by corresponding non-human residues, or by residues not present in either donor or acceptor antibodies. These modifications are made to the further refine and optimise the properties of the antibody.

For detection of endoPDI in liquid samples, such as tissue homogenates and cell lysates, the binding agent may be immobilised on a solid support, e.g. at defined, spatially separated locations, to make them easy to manipulate during the assay.

The sample is generally contacted with the binding agent(s) under appropriate conditions which allow the analyte in the sample to bind to the binding agent(s). The fractional occupancy of the binding sites of the binding agent(s), or retention of the binding agent by the sample, can then be determined either by directly or indirectly labelling the binding agent or endoPDI, or by using a developing agent or agents to arrive at an indication of the presence or amount of endoPDI in the sample. Typically, the developing agents are themselves either directly or indirectly labelled (e.g. with radioactive, fluorescent or enzyme labels, such as horseradish peroxidase) so that they can be detected using techniques well known in the art. Directly labelled developing agents have a label associated with or coupled to the agent. Indirectly labelled developing agents may be capable of binding to a labelled species (e.g. a labelled antibody capable of binding to the developing agent) or may act on a further species to produce a detectable result. Thus, radioactive labels can be detected using a scintillation counter or other radiation counting device, fluorescent labels using a laser and confocal microscope, and enzyme labels by the action of an enzyme label on a substrate, typically to produce a colour change. In further embodiments, the developing agent or analyte is tagged to allow its detection, e.g. linked to a nucleotide sequence which can be amplified in a PCR reaction to detect the analyte. Binding of a nucleic acid binding agent to endoPDI nucleic acid may be detected by nucleic acid amplification techniques, such as PCR. Other labels are known to those skilled in the art are discussed below. The developing agent(s) can be used in a competitive method in which the developing agent competes with the analyte for occupied binding sites of the binding agent, or non-competitive method, in which the labelled developing agent binds analyte bound by the binding agent or to occupied binding sites. Both methods provide an indication of the number of the binding sites occupied by the analyte, and hence the concentration of the analyte in the sample, e.g. by comparison with standards obtained using samples containing known concentrations of the analyte.

Therapeutic Methods

Binding agents as described above, and antibodies in particular, may have therapeutic potential in inhibiting angiogenesis. They may exert their effects directly, e.g. through the Fc region, by initiating innate cell-free or cell-mediated host defence mechanisms, or indirectly by targeting other therapeutic agents to the site. For example, an antibody may be labelled with an effector molecule such as a toxin molecule. or an enzyme capable of converting a prodrug to its active form. This may result in targeted killing of the cell to which the antibody is bound (typically an endothelial cell) or of neighbouring cells. (such as tumour cells).

Pharmaceuticals

Therapeutics of the invention can be formulated in pharmaceutical compositions. These compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering these agents directly, they could be produced in the target cells by expression from an encoding gene introduced into the cells, e.g. in a viral vector (a variant of the VDEPT technique—see below). The vector could be targeted to the specific cells to be treated, or it could contain regulatory elements which are switched on more or less selectively by the target cells.

Alternatively, the agent could be administered in a precursor form, for conversion to the active form by an activating agent produced in, or targeted to, the cells to be treated. This type of approach is sometimes known as ADEPT or VDEPT; the former involving targeting the activating agent to the cells by conjugation to a cell-specific antibody, while the latter involves producing the activating agent, eg an enzyme, in a vector by expression from encoding DNA in a viral vector (see for example, EP-A-415731 and WO 90/07936).

A composition may be administered alone or in combination with other treatments, either simultaneously or sequentially dependent upon the condition to be treated.

Mimetics

Once an active “lead” compound has been identified, it may be used as the basis for design of a mimetic. The designing of mimetics to a known pharmaceutically active compound is a known approach to the development of pharmaceuticals based on a “lead” compound. This might be desirable where the active compound is difficult or expensive to synthesise or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large number of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, eg by substituting each residue in turn. These parts or residues constituting the active region of the compound are known as its “pharmacophore”.

Once the pharmacophore has been found, its structure is modelled to according its physical properties, eg stereochemistry, bonding, size and/or charge, using data from a range of sources, eg spectroscopic techniques, X-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound. The mimetic or mimetics found by this approach can then be screened to see whether they have the target property, or to what extent they exhibit it. Further optimisation or modification can then be carried out to arrive at one or more final mimetics for in vivo or clinical testing.

The EndoPDI Promoter

The above description has generally been concerned with the coding portions of the endoPDI gene. Also embraced within the present invention are untranscribed parts of the gene.

Thus an aspect of the invention is an isolated nucleic acid molecule comprising the promoter of the endoPDI gene, which is capable of providing endothelial and/or hypoxia-induced gene expression.

Thus constructs comprising the endoPDI promoter may have utility in obtaining expression of therapeutic agents (such as antisense agents (including siRNA etc.), and enzymes capable of converting prodrugs to an active form, as described above) in appropriate cell types and under appropriate conditions. Alternatively they may be useful for investigation of the transcriptional regulation of the endoPDI gene, e.g. when operably coupled to a reporter gene.

The promoter region may be readily identified using standard genetic analysis techniques well known to the skilled person. This may involve in silico analysis, using programs well known in the art to identify likely transcriptional regulatory elements, and/or in vitro analysis, using well known techniques such as linker-scanning mutagenesis (see, for example, Molecular Cloning, A Laboratory Manual, 3rd edition, Sambrook and Russell, Cold Spring Harbor Laboratory Press, New York, 2001 and references cited therein.

Promoter activity is assessed using a test transcription system. “Promoter activity” is used to refer to ability to initiate transcription. The level of promoter activity is quantifiable for instance by assessment of the amount of mRNA produced by transcription from the promoter or by assessment of the amount of protein product produced by translation of mRNA produced by transcription from the promoter. The amount of a specific mRNA present in an expression system may be determined for example using specific oligonucleotides which are able to hybridise with the mRNA and which are labelled or may be used in a specific amplification reaction such as the polymerase chain reaction.

Use of a reporter gene facilitates determination of promoter activity by reference to protein production. The reporter gene preferably encodes an enzyme which catalyses a reaction which produces a detectable signal, preferably a visually detectable signal, such as a coloured product. Many examples are known, including β-galactosidase, luciferase or fluorescent proteins such as GFP. β-galactosidase activity may be assayed by production of blue colour on substrate, the assay being by eye or by use of a spectrophotometer to measure absorbance. Fluorescence, for example that produced as a result of luciferase activity, may be quantitated using a spectrophotometer. Radioactive assays may be used, for instance using chloramphenicol acetyltransferase, which may also be used in non-radioactive assays. The presence and/or amount of gene product resulting from expression from the reporter gene may be determined using a molecule able to bind the product, such as an antibody or fragment thereof. The binding molecule may be labelled directly or indirectly using any standard technique.

Those skilled in the art are well aware of a multitude of possible reporter genes and assay techniques which may be used to determine promoter activity. Any suitable reporter/assay may be used and it should be appreciated that no particular choice is essential to or a limitation of the present invention.

Also embraced by the present invention is a promoter which is a mutant, derivative, or other homologue of the endoPDI promoter, such as a promoter from one of the endoPDI orthologues described herein. These can be generated or identified as described above; they will share homology with the endoPDI promoter and retain promoter activity.

To find minimal elements or motifs responsible for tissue and/or developmental regulation, restriction enzyme or nucleases may be used to digest a nucleic acid molecule, or mutagenesis may be employed, followed by an appropriate assay (for example using a reporter gene such as luciferase) to determine the sequence required. Nucleic acid comprising these elements or motifs forms one part of the present invention.

Preferably the promoters of the present invention retain endothelial cell specificity and/or the ability to be induced by hypoxia.

In a further aspect of the invention there is provided a nucleic acid construct, preferably an expression vector, including the endoPDI promoter region or fragment, mutant, derivative or other homologue or variant thereof able to promote transcription, operably linked to a heterologous gene, e.g. a coding sequence, which is preferably not the coding sequence with which the promoter is operably linked in nature.

The promoter may be operably linked to a reporter gene for use in assays for substances capable of modulating expression of endoPDI, or for introducing markers into cells capable of expressing endoPDI, either in a diagnostic or therapeutic context. The promoter may be operably linked to nucleic acid encoding an agent capable of inhibiting endoPDI expression as described herein, or to nucleic acid encoding a substance inhibitory or lethal to a cell capable of expressing endoPDI, either directly or indirectly. For example the promoter could be use to drive expression of an enzyme capable of converting a prodrug into its active form. Such a construct could be used therapeutically by controlling activation of a prodrug only at sites of angiogenesis.

Experimental Procedures

Bioinformatic methods; EndoPDI was initially found as a gene preferentially expressed in vascular endothelial cells by analysis of expression data deposited in SAGEmap (15). Briefly, the SAGEmap data set was downloaded from the project website (www.ncbi.nlm.nih.gov/SAGE/) in February 2001 and deposited in a MySQL database. Only normal tissue libraries (total=37) were used in the analysis. There were two libraries representing vascular endothelium; SAGE_Duke_HMVEC and SAGE_Duke_HMVEC+VEGF. The preferential Expression Measure—PEM was used to identify genes preferentially expressed in vascular endothelium. PEM=log(o/e), where o is the observed SAGE tag count in vascular endothelium, and e is the expected tag count if the distribution was uniform across the libraries. e=(G*N/T), where G is the total number of SAGE tags for a given gene, N is the total number of tags for vascular endothelium (110,460), and T is the total number of tags in all normal libraries (1,077,231). The vascular endothelial PEM score for EndoPDI was 1.941. The highest vascular endothelial score yet seen is attributed to EGF-containing fibulin-like extracellular matrix protein-1 at 2.081. Von Willebrand factor (vWF) is a well characterised endothelial specific gene that had a PEM score of 1.847.

Culture of endothelial cells. Human dermal microvascular endothelial cells (HDMEC) and human umbilical vein endothelial cells (HUVEC) were purchased from Clonetics BioWhittaker (Wokingham, Berkshire, United Kingdom) and were cultured in MCDB131 medium (Gibco) containing 20% fetal calf serum (Sigma-Aldrich, Gillingham, Dorset, United Kingdom), 100 U/ml penicillin, 100 μg/ml streptomycin, 2 mM glutamine, 5 IU/ml heparin and 50 μg/ml endothelial cell growth supplement (Sigma, Dorset, UK). Cells were routinely split 1 in 3 and were used up to the 8th passage.

Isolation of full length cDNA for EndoPDI; Total RNA was extracted from HDMEC and 1 μg was reverse transcribed using Superscript II reverse transcriptase (Life Technologies, Paisley, UK). The first 270 nucleotides at the 5′ end were then amplified by PCR using the upstream primer 5° CCGGTACCCCCGCGCGCCCAGGACGCCTCCTCC-3′, designed to include a Kpn1 restriction endonuclease site, and the downstream primer 5′-GCAGCATCCAGTTTTCCAGT′-3′ and the PCR product cloned into the topo II PCR vector (Life Technologies) and sequenced. An IMAGE clone (3356029) containing partial EndoPDI cDNA sequence was identified from the Unigene database. The clone is complete at the 3′ end but is truncated by 255 nucleotides at the 5′ end. The unique BstUI site at position 267 was used to enable the ligation of the IMAGE clone EndoPDI insert with the cloned 270 nucleotides of the 5′ end to give a full-length cDNA of EndoPDI.

Production of polyclonal antibodies to EndoPDI and Western blotting; The peptide of sequence ADGEDGQDPHSKC was synthesized by the Protein and Peptide Chemistry Department of Cancer Research UK, using standard techniques. This sequence corresponds to amino acids 52 to 63 of the human EndoPDI protein sequence with an additional cysteine residue added to the C-terminus to enable coupling to a carrier protein. The peptide was coupled to Imject® Maleimide Activated mcKLH (Pierce, Rockford, USA) following the manufacturer's instructions and diluted with Freunds adjuvant before injection into rabbits. A standard immunisation protocol was followed with 200 μg immunogen used for the first injection and 100 μg immunogen for subsequent boosts. The test bleeds were screened against pre-bleeds by ELISA to identify the presence of antibodies to EndoPDI in the rabbit serum. Serum that was found to contain EndoPDI antibodies by ELISA was used at 1/100 dilution in Western blotting experiments for the detection of EndoPDI protein by standard Western blotting techniques.

Preparation of a recombinant EndoPDI construct for riboprobe synthesis. A 300 bp region of the 3′UTR of EndoPDI was amplified by PCR from plasmid DNA containing the EndoPDI clone described above using 5′-TGTGGCTCCTGAGTTGAGTG-3′ as the upstream forward primer and 5′-ACTCAGGCACGGTCAGAAGT-3′ as the reverse downstream primer. The Basic Local Alignment Search Tool (BLAST) (16) was used to ensure that the chosen region of EndoPDI did not have homology to other sequences. The PCR product was cloned into PCRII-TOPO (Invitrogen, Paisley, UK) following the manufacturers' instructions and sequenced to confirm identity and orientation. Riboprobes were transcribed (MAXIscript in vitro transcription kit, Ambion AMS Ltd, Witney, Oxon, United Kingdom) from linearised plasmids in the presence of [32P]UTP (Amersham Pharmacia, Amersham, UK) to give radioactively labelled probe.

RNase Protection Assay. Total RNA was extracted from cells in culture using TRI reagent (Sigma, Poole, Dorset, UK). RNase protection assays were performed in duplicate on 10-30 μg of total RNA as described (16). To attenuate the signal strength of the highly abundant loading control, U6 small nuclear RNA (accession no. X01366), a riboprobe of significantly lower specific activity was prepared by addition of unlabeled CTP to the labelling reaction. The protected fragment size for EndoPDI was 300 nucleotides. In each assay, a positive and negative (tRNA only) control and undigested riboprobes were analysed. Intensity of signal, quantified on a PhosphorImager (Molecular Dynamics, Chesham, Buckinghamshire, United Kingdom) was calculated as the ratio of the signal of interest to U6 mRNA to correct for variations in loading.

In situ hybridisation: In situ hybridisation analysis was performed with radioactively labelled probes as described in Poulsom et al., 1998 (17). The EndoPDI specific probe used for in situ hybridisation was the same as that used for the RNase protection assay described above. Human tissue was collected with full ethical approval during routine pathology, fixed in formalin and embedded in paraffin.

Multiple Tissue Array Analysis: Human multiple tissue expression arrays (Clontech, Oxford, UK) with poly(A)+ RNA from different tissues were used for analysis of the distribution of EndoPDI mRNA in human tissues. DNA from the same region of EndoPDI used for riboprobe production was used for preparation of a cDNA probe. The cDNA was labelled with [32P]dCTP (Rediprime random primer labelling kit, Amersham) and hybridised at 65° C. overnight in ExpressHyb (Clontech) solution following manufacturer's instructions.

Transfection of microvascular endothelial cells with siRNA: EndoPDI specific (5′-GAAGCTGTGAAGTACCAGGTT-3′) and PDI specific (5′-GACCTCCCCTTCAAAGTTGTT-3′) siRNA oligos were synthesized using the SilencerTM siRNA Construction kit (Ambion, Huntingdon, UK) following manufacturer's instructions. HDMEC (5×105 cells) were plated onto 0.2% gelatin coated 10 cm petri dishes and incubated for 24 h. Cells were then transfected with 10 nM EndoPDI and/or PDI siRNA using oligofectamine reagent (Invitrogen) according to the manufacturer's instructions. The cells were then incubated for 24 h prior to hypoxia for 16 h (0.1% O2, 5% CO2 and 94.9% N2) or continuation of normoxic exposure for 16 h before performing FACS analysis as described.

FACS Analysis: To distinguish apoptotic from necrotic cells, double staining for exposed phosphatidylserine and propidium iodide (PI) exclusion was performed as follows: Cells were harvested, washed twice with PBS and resuspended in binding buffer (10 mM HEPES/NaOH, pH 7.4, 140 mM NaCl, 2.5 mM CaC12). Five μl Annexin V-FITC antibody (Pharmingen, San Diego, USA) and 10 μl PI (50 μg/ml) were added to the cells. After incubation for 15 min in the dark at room temperature, the cells were analysed by a FACScan. Controls of unstained cells, cells stained with Annexin V-FITC only and cells stained with PI only were used to set up compensation and quadrants.

The cell surface expression of CD105 was quantified by incubating 105 cells per tube with 50 μl (10 μl/ml in PBS) of monoclonal antibody to CD105 on ice for 1 h and washed twice with cold PBS. After incubation with FITC-labelled rabbit anti-mouse F(ab)2 (1/40 DAKO) for 30 min on ice, the cells were washed and resuspended in 0.3 ml of 2% buffered formalin and analyzed on a FACScan.

Measurement of endothelin-1 secretion by ELISA: The secretion of endothelin-1 by endothelial cells was measured using a human endothelin-1 ELISA (R&D Systems, Abindgdon, UK) following manufacturer's instructions. Briefly, 50,000 cells were treated with RNAi oligos as before, the medium collected and diluted 1/25 for use in the ELISA.

Measurement of adrenomedullin by Radioimmunoassay: The secretion of adrenomedullin by endothelial cells was measured using an adrenomedullin radioimmunoassay (Peninsula Laboratories Europe, Ltd, Merseyside, England) following manufacturer's instructions. Briefly, 50,000 cells were treated with RNAi oligos as before, the medium collected and used in the radioimmunoassay.

Results

Identification of EndoPDI using bioinformatic and cDNA sequence analysis. We utilized serial analysis of gene expression (SAGE) libraries (http://www.ncbi.nlm.nih.gov/SAGE) to find genes that are highly expressed in endothelial cells. Using this approach we identified a novel gene, which we subsequently called EndoPDI, that is highly expressed in both VEGF stimulated and quiescent microvascular endothelial cells (HMVEC) with counts of 1224 and 741 tags per million respectively (table 1). The library with the next highest tag count for EndoPDI was the foreskin fibroblast library with 204 tags per million, i.e., less than a third of that for HMVEC. Homologues of EndoPDI have been identified in the mouse, rat, Xenopus, Drosophila and mosquito (FIG. 1A). While PDI has only two APWCGHC thioredoxin motifs, EndoPDI has three (FIG. 1B), however, both have in common a C-terminal KDEL sequence which is characteristic of proteins that are retained within the endoplasmic reticulum. We used the Neural Network programme, SignalIP (18) to analyse the protein sequence of EndoPDI and found that it contains a signal peptide, MPARPGRLLPLLARPAALTALLLLLLGHGGGGRW at the N-terminus with the most likely cleavage site being located between positions 32 and 33 (GGG-RW). All other members of the PDI family contain a domain that has the thioredoxin structural fold, also called ‘b’ domain, but contain no thioredoxin active site. Using the structure prediction programme VAST (19) we found that EndoPDI has the structure ao, a, a′, c with no b domain (FIG. 1B).

Genomic organisation, chromosomal localisation and tissue distribution of EndoPDI. The putative full-length gene encoding EndoPDI has been found in genome databases (Accession No. BD127641) and mapped to human chromosome 6 at position 6p25.2. This region of chromosome 6 also encodes another molecule containing thioredoxin-like domains called PICOT (20).

We performed RNase protection analysis to examine the expression of EndoPDI in vitro in ten different cell types (FIG. 2). As expected, EndoPDI expression was highest in endothelial cells. The other cell lines tested were MRC-5 (fibroblast), SY—SH—SY (neuroblastoma), SK23 (skin fibroblast), MDA468 (breast carcinoma), NCIH520 (squamous lung carcinoma), ZR75 (oestrogen dependent breast carcinoma) and HL60 (promyelocytic leukemia). We found that EndoPDI expression was greatest in large vessel endothelial cells (HUVEC) compared with microvascular endothelial cells (HDMEC), with the immortalised cell line HMME displaying the lowest expression among the endothelial cells. The promyelocytic leukemia cell line, HL60 displayed a 2.5 fold higher expression of EndoPDI than MDA468, the next highest expressing cell line. Expression of EndoPDI in HL60 may reflect the common cell lineage of haemotopoetic and endothelial cells in that both originate from haemangioblasts in the embryonic blood islands. The expression of EndoPDI in the remaining 6 cell lines was at least 4 fold lower than in endothelial cells.

Tissue Expression Array studies: Human multiple tissue expression arrays were used to determine the relative expression of EndoPDI mRNA in human tissues (FIG. 3A). The blots contained poly A+ RNA from 72 different human tissues. EndoPDI was detected in 20 out of the 72 tissue spots. The highest levels were found in lymph node, followed by stomach then heart. The high expression in the stomach was unexpected since the stomach is not a particularly well vascularised organ, and in light of the hypoxic induction of EndoPDI described later, not known to be hypoxic. Arrays containing tissue from matched tumor and normal tissue samples were used to determine whether EndoPDI is upregulated in human tumors (FIG. 3B). Up-regulation of endoPDI was found in tumors of the cervix, uterus, stomach and lung.

In situ hybridisation studies: In situ hybridisation studies were performed in order to define expression of EndoPDI in human tissues in vivo (FIG. 4). Expression was found to be rare and seen only in the vasculature of human melanoma (A-D), the syncytiotrophoblasts of placenta (E and F), in macrophages and the microvasculature of the atherosclerotic plaque (G and H) and in the keratinocytes of a hair follicle (I and J).

EndoPDI is up-regulated by hypoxia in HDMEC. We next investigated whether EndoPDI is regulated by hypoxia. Using RNase protection analysis we found that EndoPDI is 2-fold upregulated after 1 h hypoxia in HDMEC with a maximal 2.5-fold induction after 16 h hypoxia (FIG. 5A). Western analysis confirmed the protein to be 3-fold up-regulated by hypoxia (FIG. 5B).

Loss of EndoPDI causes increased apoptotic cell death in microvascular endothelial cells in hypoxia but not normoxia. Since the up-regulation of PDI has been previously shown to have a protective effect against apoptotic cell death induced by hypoxia in neuronal cells (21), we investigated whether EndoPDI has a role in protecting endothelial cells from apoptosis under hypoxia. The approach we used was to down-regulate EndoPDI expression using specific siRNA oligos. The siRNA efficiently down-regulated EndoPDI mRNA (FIG. 6A) and protein (FIG. 6B). Cells were treated with either transfection reagents alone, EndoPDI specific siRNA or scrambled siRNA. Scrambled siRNA is siRNA that contains the same overall nucleotide composition as the gene specific siRNA but has no homology to any known genes according to BLAST search results. We found that under normoxia, HDMEC that had been treated with transfection reagents alone (control) or with scrambled oligos expressed the same level of EndoPDI mRNA and protein and this expression was completely blocked after transfection with EndoPDI specific siRNA (FIG. 6). Similar results were observed when the cells were subjected to 16 h hypoxia (0.1% O2) in that the expression level of EndoPDI mRNA and protein was unaffected by treatment with scrambled siRNA (FIG. 6). Again under hypoxia, there was loss of EndoPDI expression after transfection with siRNA specific to EndoPDI.

We used siRNA to examine the effect of down-regulation of EndoPDI on HDMEC survival under hypoxia. We found that down-regulation of EndoPDI under normoxia had no effect on HDMEC survival (FIG. 7A). However, when HDMEC were treated with EndoPDI siRNA under hypoxia there was a significant increase in the apoptotic and necrotic cell populations (FIG. 7B).

Loss of PDI causes increases apoptotic cell death in microvascular endothelial cells in both normoxia and hypoxia. The effect of EndoPDI on endothelial cell behaviour was compared to that of PDI. Using PDI specific siRNA to down-regulate PDI, we performed FACS analysis to determine the extent of apoptosis resulting from the loss of PDI (FIG. 8). In contrast to EndoPDI, we found that loss of PDI caused a high level of apoptosis in normoxia as well as under hypoxia. In fact, under the same conditions, PDI down-regulation resulted in 55% and 48% of the cell population being apoptotic in normoxia and hypoxia respectively whereas 12% of the cell population was apoptotic under hypoxia after down-regulation of EndoPDI and only 4.5% apoptotic under normoxia.

The effect of lack of EndoPDI and PDI expression on the secretion or cell surface expression of hypoxically induced endothelial survival factors. Under hypoxia, endothelial cells produce a number of molecules that act as hypoxia survival factors. Examples of such molecules are endothelin-1 (22) adrenomedullin (23) and CD105 (24). We compared the expression of these molecules by endothelial cells under hypoxia after knockout of EndoPDI, PDI or both EndoPDI and PDI together. There was a significant decrease in endothelin-1 expression after PDI siRNA treatment but an even greater reduction after EndoPDI siRNA treatment (FIG. 9A). The reduction in endothelin-1 secretion in the absence of EndoPDI was equal to the secretion of endothelin-1 under normoxia. Further, treatment with combined EndoPDI and PDI siRNA caused a larger reduction in the secretion of endothelin-1 compared to that of EndoPDI siRNA alone. These results suggest that both PDI and EndoPDI have a role in the folding and secretion of endothelin-1 under hypoxia and that EndoPDI has a greater specificity for endothelin-l than PDI.

In contrast, while loss of EndoPDI expression significantly reduced adrenomedullin secretion under hypoxia, loss of PDI expression had little effect. Furthermore, there was no significant difference in adrenomedullin secretion after both PDI and EndoPDI siRNA treatment compared with EndoPDI siRNA alone. These results suggest that PDI has little or no role in the folding and secretion of adrenomedullin under hypoxia, but that EndoPDI has greater specificity for this molecule. That there is still adrenomedullin production after the knockout of both PDI and EndoPDI suggests that there are other chaperones that function in the folding and secretion of this peptide.

CD105 (also called endoglin) is an endothelial specific gene whose expression is upregulated by hypoxia and has been shown to protect endothelial cells from apoptosis under hypoxia (24). FIG. 9C shows that hypoxia caused up-regulation of CD105 expression on HDMEC, corresponding to a 64% increase in cell surface expression as determined using the geometric mean fluorescent intensities. While siRNA to PDI had little effect on CD105 expression, that to EndoPDI caused a marked reduction in expression of 50% of the population (two peaks apparent) and that the two siRNAs administered together completely ablated CD105 expression. The effect of EndoPDI specific siRNA on CD105 expression is similar to that previously reported for treatment with antisense CD105 where a similar CD105 mixed cell expression population results (24). We conclude that while EndoPDI may complement CD105 folding in the absence of PDI, the reverse is not the case, arguing strongly for a role for EndoPDI in folding this molecule under hypoxia.

Discussion

We describe here a novel human protein disulphide isomerase that we have called EndoPDI due to its high and preferential expression in endothelial cells. Experiment has shown that EndoPDI expression is upregulated by hypoxia, is only expressed in vivo in hypoxic tissues and protects endothelial cells from death under hypoxia. We also show that the protective effect of EndoPDI under hypoxia could be due to a folding and chaperone activity on hypoxically induced anti-apoptotic molecules.

Unlike archetypal PDI, EndoPDI is a rare example of a PDI that has three a-type domains containing the conserved APWCGHC thioredoxin domain but no b domains. The existence of a strong Kozak sequence and a well defined signal peptide sequence lends support to this being a full length cDNA clone for EndoPDI. The N-terminal leader sequence and the C-terminal KDEL, provide strong evidence that like PDI, EndoPDI should be predominantly localised to the ER.

EndoPDI appears to have an unusual pattern of tissue expression. As expected we detected expression in well vascularised tissues such as heart, lung and lymph node but unexpectedly we found higher expression in some tissues of comparatively low vascular density such as the stomach. Nevertheless, the overall pattern of tissue expression obtained for EndoPDI from the tissue expression array is fairly consistent with the SAGE expression data; for example the SAGE data suggests that the expression of EndoPDI in the stomach is around 4-fold higher than in the heart. Furthermore, the SAGE array data suggests that the expression of EndoPDI in the liver and heart is roughly equal, a result that is confirmed by the tissue expression array data. The relative levels of the other PDI family members vary greatly between different tissue types, between different cell types within the same tissue and with the physiological state of the cell (28). The only member of the PDI family to date shown to have restricted tissue expression is PDIp, the pancreas specific PDI (29). PDIp may be required for the folding of pancreas specific proteins such as zymogens (30). We may similarly speculate that EndoPDI may be required for the folding of endothelial cell specific proteins.

Expression of EndoPDI in endothelial cells is at least 3 fold higher than in the carcinoma or other tumor cell lines tested, with the notable exception of HL60. A detailed array analysis recently identified EndoPDI (known as MGC3178 in that study) as the most upregulated gene in multiple myeloma (31). In view of this, and the fact that HL60 cells are derived from the same embryonic lineage as endothelial cells, expression in HL60 cells was not surprising.

In situ analysis showed EndoPDI to be primarily expressed in areas of known or putative hypoxia, notably tumors (32) atherosclerotic plaques (33) and the bulb of a hair follicle (34). Thus, in tumors EndoPDI was present in the vascular endothelium (FIGS. 4A-D), in atherosclerotic plaques in macrophages and microvascular endothelium of the lesion (FIGS. 4G and H) and in keratinocytes of the hair bulb (FIGS. 4I and J). Unexpectedly, expression was also seen in the syncytiotrophoblasts of the normal placenta. From these observations it seems reasonable to conclude that expression of EndoPDI in vivo is primarily a result of oxygen deprivation.

Endothelial cells are the most transcriptionally active cell type yet identified (35) and express many proteins that are specific to or relatively highly expressed in them, for example Delta 4, Robo4, E-Cadherin, von-Willebrand factor, KDR, Tie-1, Tie-2, CD31 and CD105 (Huminiecki and Bicknell, 2000; Ho et al, 2003). Furthermore, unlike other cell types, endothelial cells are able to withstand considerable stress such as hypoxia, prolonged glucose deprivation and sustained heat shock (36). This is of particular significance in the survival and resistance to cytotoxic drugs of tumor vessels and presents an obstacle to some anti-cancer drugs that damage the vasculature. There are a unique set of proteins found to be expressed in endothelial cells during hypoxia (37) and it is conceivable that EndoPDI might be essential for the folding and export of these additional proteins required for survival under hypoxia. Archetypal PDI is also upregulated during hypoxia in endothelial cells (38) as well as in glial cells (21). The up-regulation of PDI in glial cells was shown to have a protective effect against hypoxic cell death in these cells (21). Our results show that PDI is an absolute requirement of endothelial cell survival under both normoxia and hypoxia, but that EndoPDI is highly expressed in and protects endothelial cells under hypoxia. We therefore hypothesised that EndoPDI in concert with PDI enables endothelial cells to show a greater ability for survival under hypoxia. To test this hypothesis, we measured the secretion of endothelin-1 and adrenomedullin together with the cell surface expression of CD105, molecules with known protective effects against hypoxia-induced apoptosis in endothelial cells. All three are known to be hypoxically induced genes and to protect endothelial cells from death under hypoxia (22,24,27). Knockout of EndoPDI markedly downregulated expression of all three proteins. Knockout of PDI alone had no effect on CD105 expression whereas knockout of EndoPDI and PDI completely abolished expression.

In summary, we have identified EndoPDI a novel protein disulphide isomerase-like protein that is highly expressed in endothelial cells, is upregulated by hypoxia and is expressed in the endothelium of tumors and atherosclerotic plaques. EndoPDI appears to be a requirement for endothelial cell survival under hypoxic conditions due to its enabling secretion of several endothelial cell survival factors. Further work is needed to delineate the role of PDI and EndoPDI in the hypoxic endothelial cell.

Abbreviations

Endoplasmic Reticulum, ER; Endothelial Protein Disulphide Isomerase, EndoPDI; Human Umbilical Vein Endothelial Cells, HUVEC; Human Dermal Microvascular Endothelial Cells, HDMEC; Fluorescence Activated Cell Sorting, FACS; Polymerase Chain Reaction, PCR; short interfering RNA, siRNA; von Willebrand Factor, vWF.

References

All references provided below and cited elsewhere herein are expressly incorporated by reference.
1. Freedman, R. B., Hirst, T. R., and Tuite, M. F. (1994) Trends Biochem Sci 19, 331-336
2. Gething, M. J., and Sambrook, J. (1992) Nature 355, 33-45
3. Edman, J. C., Ellis, L., Blacher, R. W., Roth, R. A., and Rutter, W. J. (1985) Nature 317, 267-270
4. Hawkins, H. C., and Freedman, R. B. (1991) Biochem J 275 ( Pt 2), 335-339
5. LaMantia, M. L., and Lennarz, W. J. (1993) Cell 74, 899-908
6. Lyles, M. M., and Gilbert, H. F. (1994) J Biol Chem 269, 30946-30952
7. Darby, N. J., and Creighton, T. E. (1995) Biochemistry 34, 11725-11735
8. Yao, Y., Zhou, Y., and Wang, C. (1997) Embo J 16, 651-658
9. Koivunen, P., Pirneskoski, A., Karvonen, P., Ljung, J., Helaakovski, T., Notbohm, H., and Kivirikko, K. I. (1999) EMBO J 18, 65-74
10. John, D. C., Grant, M. E., and Bulleid, N. J. (1993) EMBO J 12, 1587-1595
11. McLaughlin, S. H., and Bulleid, N. J. (1998) Biochem J 331 ( Pt 3), 793-800
12. Clissold, P. M., and Bicknell, R. (2003) Bioessays 25, 603-611
13. Elliott, J. G., Oliver, J. D., and High, S. (1997) J Biol Chem 272, 13849-13855
14. Oliver, J. D., van der Wal, F. J., Bulleid, N. J., and High, S. (1997) Science 275, 86-88
15. Lash, A. E., Tolstoshev, C. M., Wagner, L., Schuler, G. D., Strausberg, R. L., Riggins, G. J., and Altschul, S. F. (2000) Genome Res 10, 1051-1060
16. Altschul, S. F., Madden, T. L., Schaffer, A. A., Zhang, J., Zhang, Z., Miller, W., and Lipman, D. J. (1997) Nucleic Acids Res 25, 3389-3402
17. Poulsom, R., Longcroft, J. M., Jeffery, R. E., Rogers, L. A., and Steel, J. H. (1998) Eur J Histochem 42, 121-132
18. Nielsen, H., Engelbrecht, J., Brunak, S., and von Heijne, G. (1997) Protein Eng 10, 1-6
19. Gibrat, J. F., Madej, T., and Bryant, S. H. (1996) Curr Opin Struct Biol 6, 377-385
20. Witte, S., Villalba, M., Bi, K., Liu, Y., Isakov, N., and Altman, A. (2000) J Biol Chem 275, 1902-1909
21. Tanaka, S., Uehara, T., and Nomura, Y. (2000) J Biol Chem 275, 10388-10393
22. Shichiri, M., Kato, H., Marumo, F., and Hirata, Y. (1997) Hypertension 30, 1198-1203
23. Oehler, M. K., Norbury, C., Hague, S., Rees, M. C., and Bicknell, R. (2001) Oncogene 20, 2937-2945
24. Li, C., Issa, R., Kumar, P., Hampson, I. N., Lopez-Novoa, J. M., Bernabeu, C., and Kumar, S. (2003) J Cell Sci 116, 2677-2685
25. Kourembanas, S., Marsden, P. A., McQuillan, L. P., and Faller, D. V. (1991) J Clin Invest 88, 1054-1057
26. Sugo, S., Minamino, N., Kangawa, K., Miyamoto, K., Kitamura, K., Sakata, J., Eto, T., and Matsuo, H. (1994) Biochem Biophys Res Commun 201, 1160-1166
27. Miller, M. J., Martinez, A., Unsworth, E. J., Thiele, C. J., Moody, T. W., Elsasser, T., and Cuttitta, F. (1996) J Biol Chem 271, 23345-23351
28. Freedman, R. B., Dunn, A. D., and Ruddock, L. W. (1998) Curr Biol 8, R468-470
29. Desilva, M. G., Notkins, A. L., and Lan, M. S. (1997) DNA Cell Biol 16, 269-274
30. Volkmer, J., Guth, S., Nastainczyk, W., Knippel, P., Klappa, P., Gnau, V., and Zimmermann, R. (1997) FEBS Lett 406, 291-295
31. Claudio, J. O., Masih-Khan, E., Tang, H., Goncalves, J., Voralia, M., Li, Z. H., Nadeem, V., Cukerman, E., Francisco-Pabalan, O., Liew, C. C., Woodgett, J. R., and Stewart, A. K. (2002) Blood 100, 2175-2186
32. Vaupel, P., Kallinowski, F., and Okunieff, P. (1990) Adv Exp Med Biol 277, 895-905
33. Bjornheden, T., Levin, M., Evaldsson, M., and Wiklund, O. (1999) Arterioscler Thromb Vasc Biol 19, 870-876
34. Yano, K., Brown, L. F., and Detmar, M. (2001) J Clin Invest 107, 409-417
35. Adams, M. D., Kerlavage, A. R., Fleischmann, R. D., Fuldner, R. A., Bult, C. J., Lee, N. H., Kirkness, E. F., Weinstock, K. G., Gocayne, J. D., White, O., and et al. (1995) Nature 377, 3-174
36. Tucci, M., McDonald, R. J., Aaronson, R., Graven, K. K., and Farber, H. W. (1996) Am J Physiol 271, L341-348
37. Graven, K. K., and Farber, H. W. (1998) J Lab Clin Med 132, 456-463
38. Graven, K. K., Molvar, C., Roncarati, J. S., Klahn, B. D., Lowrey, S., and Farber, H. W. (2002) Am J Physiol Lung Cell Mol Physiol 282, L996-1003
39. Freedman, R. B., Klappa, P., and Ruddock, L. W. (2002) EMBO Reports 3(2), 136-140.

1. A method for testing the ability of a candidate substance to modulate endoPDI activity, comprising contacting endoPDI with the candidate substance and determining the effect of the candidate substance on endoPDI activity.

2. A method according to claim 1 comprising assessing formation or disruption of a disulphide bond in a reporter molecule or molecules.

3. A method according to claim 2 comprising assessing rearrangement of disulphide bonds in a reporter molecule.

4. A method according to claim 1 further comprising contacting a control protein comprising a thioredoxin domain with the candidate substance and determining the effect of the candidate substance on the activity of the control protein.

5. A method according to claim 4, wherein said control protein is a protein disulphide isomerase other than endoPDI.

6. A method according to claim 2 wherein said reporter molecule is an enzyme.

7. A method according to claim 6 wherein the reporter molecule is an RNase or DNase.

8. A method according to claim 1 comprising contacting endoPDI with a library of candidate substances and selecting a substance capable of modulating endoPDI activity.

9. A method for testing the ability of a candidate substance to modulate endoPDI expression, comprising contacting a cell capable of expressing endoPDI with the candidate substance.

10. A method according to claim 9, comprising determining the effect of the candidate substance on endoPDI expression.

11. A method according to claim 9 comprising determining the effect of the candidate substance on the viability of the cell.

12. A method according to claim 11 comprising assessing apoptosis in the cell.

13. A method according to claim 9, wherein said cell is an endothelial cell or myeloma cell.

14. A method according to claim 9 wherein said cell is hypoxic.

15. A method according to claim 9 comprising causing said candidate substance to be expressed by said cell.

16. A method according to claim 15 wherein said candidate substance is an antisense agent.

17. A method according to claim 9 further comprising determining the effect of the candidate substance on the expression of a control protein.

18. A method according to claim 17 wherein the control protein comprises a thioredoxin domain.

19. A method according to claim 18 wherein the control protein is a protein disulphide isomerase other than endoPDI.

20. A substance identified by a method according to claim 1.

21. A method of formulating a pharmaceutical comprising, having identified an endoPDI antagonist by a method according to claim 1, formulating the antagonist with a pharmaceutically acceptable carrier.

22. A method according to claim 21 further comprising optimising the endoPDI antagonist for pharmaceutical administration.

23. A method of inhibiting angiogenesis comprising contacting an endothelial cell with an endoPDI antagonist or binding agent.

24. A method for testing the ability of a candidate substance to inhibit angiogenesis, comprising contacting an endothelial cell with the candidate substance, wherein the candidate substance has previously been identified as an inhibitor of endoPDI activity or expression by a method according to claim 1.

25-33. (canceled)
